SEQUENCE LISTING (150 sequences)

SEQ ID NO: 1 - DNA, 20 bp, Artificial Sequence
  sgRNA coding sequence
ggaccagccactcaccatcc

SEQ ID NO: 2 - DNA, 20 bp, Artificial Sequence
  sgRNA coding sequence
agcttcatcttccggatctt

SEQ ID NO: 3 - DNA, 20 bp, Artificial Sequence
  sgRNA coding sequence
tccttgtgactactcacctt

SEQ ID NO: 4 - DNA, 20 bp, Artificial Sequence
  sgRNA coding sequence
aactcatactcccgcacctg

SEQ ID NO: 5 - DNA, 21 bp, Artificial Sequence
  primer used for RT-PCR
ctgggtcttgtctgtctggaa

SEQ ID NO: 6 - DNA, 21 bp, Artificial Sequence
  primer used for RT-PCR
ctggtgtttacattcagcccc

SEQ ID NO: 7 - DNA, 20 bp, Artificial Sequence
  primer used for RT-PCR
ggccagaagaaccaactcca

SEQ ID NO: 8 - DNA, 20 bp, Artificial Sequence
  primer used for RT-PCR
gacagtgccacagcccttag

SEQ ID NO: 9 - DNA, 20 bp, Artificial Sequence
  primer used for RT-PCR
tcaagatggcgtgtgggatt

SEQ ID NO: 10 - DNA, 20 bp, Artificial Sequence
  primer used for RT-PCR
gaccagcaaatggtgaagcc

SEQ ID NO: 11 - DNA, 20 bp, Artificial Sequence
  primer used for RT-PCR
gatcctttggcatccggaga

SEQ ID NO: 12 - DNA, 20 bp, Artificial Sequence
  primer used for RT-PCR
gctgattctgtgtttggccc

SEQ ID NO: 13 - DNA, 20 bp, Artificial Sequence
  sgRNA coding sequence
aaaaccacggcggatggcag

SEQ ID NO: 14 - DNA, 20 bp, Artificial Sequence
  sgRNA coding sequence
tagcccaaaaccacggcgga

SEQ ID NO: 15 - DNA, 20 bp, Artificial Sequence
  sgRNA coding sequence
cccctagcccaaaaccacgg

SEQ ID NO: 16 - DNA, 20 bp, Artificial Sequence
  sgRNA coding sequence
gtgcccctagcccaaaacca

SEQ ID NO: 17 - DNA, 20 bp, Artificial Sequence
  sgRNA coding sequence
cccgcagccttccagtgaag

SEQ ID NO: 18 - DNA, 20 bp, Artificial Sequence
  sgRNA coding sequence
ccccgcagccttccagtgaa

SEQ ID NO: 19 - DNA, 20 bp, Artificial Sequence
  sgRNA coding sequence
cccccgcagccttccagtga

SEQ ID NO: 20 - DNA, 20 bp, Artificial Sequence
  sgRNA coding sequence
acctgtataactggagggac

SEQ ID NO: 21 - DNA, 20 bp, Artificial Sequence
  sgRNA coding sequence
cagaaacctgtataactgga

SEQ ID NO: 22 - DNA, 20 bp, Artificial Sequence
  sgRNA coding sequence
ccagaaacctgtataactgg

SEQ ID NO: 23 - DNA, 20 bp, Artificial Sequence
  sgRNA coding sequence
tggccagaaacctgtataac

SEQ ID NO: 24 - DNA, 20 bp, Artificial Sequence
  sgRNA coding sequence
cggaaagagagaacgggctg

SEQ ID NO: 25 - DNA, 20 bp, Artificial Sequence
  sgRNA coding sequence
tccggaaagagagaacgggc

SEQ ID NO: 26 - DNA, 20 bp, Artificial Sequence
  sgRNA coding sequence
gcaaagcgagctcaccatga

SEQ ID NO: 27 - DNA, 20 bp, Artificial Sequence
  sgRNA coding sequence
taatccgctgccatccgccg

SEQ ID NO: 28 - DNA, 20 bp, Artificial Sequence
  sgRNA coding sequence
gctgccatccgccgtggttt

SEQ ID NO: 29 - DNA, 20 bp, Artificial Sequence
  sgRNA coding sequence
ctgccatccgccgtggtttt

SEQ ID NO: 30 - DNA, 20 bp, Artificial Sequence
  sgRNA coding sequence
atccgccgtggttttgggct

SEQ ID NO: 31 - DNA, 20 bp, Artificial Sequence
  sgRNA coding sequence
tccgccgtggttttgggcta

SEQ ID NO: 32 - DNA, 20 bp, Artificial Sequence
  sgRNA coding sequence
gttttgggctaggggcacgc

SEQ ID NO: 33 - DNA, 20 bp, Artificial Sequence
  sgRNA coding sequence
ttgggctaggggcacgctgg

SEQ ID NO: 34 - DNA, 20 bp, Artificial Sequence
  sgRNA coding sequence
tgggctaggggcacgctgga

SEQ ID NO: 35 - DNA, 20 bp, Artificial Sequence
  sgRNA coding sequence
tcatgtgtttgccccttcac

SEQ ID NO: 36 - DNA, 20 bp, Artificial Sequence
  sgRNA coding sequence
gccccttcactggaaggctg

SEQ ID NO: 37 - DNA, 20 bp, Artificial Sequence
  sgRNA coding sequence
tcccgtccctccagttatac

SEQ ID NO: 38 - DNA, 20 bp, Artificial Sequence
  sgRNA coding sequence
atcatggtgagctcgctttg

SEQ ID NO: 39 - DNA, 20 bp, Artificial Sequence
  sgRNA coding sequence
tgagctcgctttgcggcgtt

SEQ ID NO: 40 - DNA, 20 bp, Artificial Sequence
  sgRNA coding sequence
gagctcgctttgcggcgttc

SEQ ID NO: 41 - DNA, 20 bp, Artificial Sequence
  sgRNA coding sequence
agctcgctttgcggcgttcg

SEQ ID NO: 42 - DNA, 20 bp, Artificial Sequence
  sgRNA coding sequence
cgctttgcggcgttcggggc

SEQ ID NO: 43 - DNA, 20 bp, Artificial Sequence
  sgRNA coding sequence
gacaagacccagcggctccc

SEQ ID NO: 44 - DNA, 20 bp, Artificial Sequence
  sgRNA coding sequence
tccagacagacaagacccag

SEQ ID NO: 45 - DNA, 20 bp, Artificial Sequence
  sgRNA coding sequence
cttgaggcatccccaggcca

SEQ ID NO: 46 - DNA, 20 bp, Artificial Sequence
  sgRNA coding sequence
gccccgcttgaggcatcccc

SEQ ID NO: 47 - DNA, 20 bp, Artificial Sequence
  sgRNA coding sequence
tttacattcagccccgcttg

SEQ ID NO: 48 - DNA, 20 bp, Artificial Sequence
  sgRNA coding sequence
atgtacgtcgtaagttgttc

SEQ ID NO: 49 - DNA, 20 bp, Artificial Sequence
  sgRNA coding sequence
ttccggatcttagggtgggg

SEQ ID NO: 50 - DNA, 20 bp, Artificial Sequence
  sgRNA coding sequence
atcttccggatcttagggtg

SEQ ID NO: 51 - DNA, 20 bp, Artificial Sequence
  sgRNA coding sequence
catcttccggatcttagggt

SEQ ID NO: 52 - DNA, 20 bp, Artificial Sequence
  sgRNA coding sequence
tcatcttccggatcttaggg

SEQ ID NO: 53 - DNA, 20 bp, Artificial Sequence
  sgRNA coding sequence
gcttcatcttccggatctta

SEQ ID NO: 54 - DNA, 20 bp, Artificial Sequence
  sgRNA coding sequence
gccactcaccatccgggaaa

SEQ ID NO: 55 - DNA, 20 bp, Artificial Sequence
  sgRNA coding sequence
agccactcaccatccgggaa

SEQ ID NO: 56 - DNA, 20 bp, Artificial Sequence
  sgRNA coding sequence
tggaccagccactcaccatc

SEQ ID NO: 57 - DNA, 20 bp, Artificial Sequence
  sgRNA coding sequence
gccgctgggtcttgtctgtc

SEQ ID NO: 58 - DNA, 20 bp, Artificial Sequence
  sgRNA coding sequence
tgggtcttgtctgtctggaa

SEQ ID NO: 59 - DNA, 20 bp, Artificial Sequence
  sgRNA coding sequence
gtcttgtctgtctggaaggg

SEQ ID NO: 60 - DNA, 20 bp, Artificial Sequence
  sgRNA coding sequence
ggcctggggatgcctcaagc

SEQ ID NO: 61 - DNA, 20 bp, Artificial Sequence
  sgRNA coding sequence
gcctggggatgcctcaagcg

SEQ ID NO: 62 - DNA, 20 bp, Artificial Sequence
  sgRNA coding sequence
atcctccccaccctaagatc

SEQ ID NO: 63 - DNA, 20 bp, Artificial Sequence
  sgRNA coding sequence
tccctttcccggatggtgag

SEQ ID NO: 64 - DNA, 20 bp, Artificial Sequence
  sgRNA coding sequence
tttcccggatggtgagtggc

SEQ ID NO: 65 - DNA, 20 bp, Artificial Sequence
  sgRNA coding sequence
agtggctggtccagagagca

SEQ ID NO: 66 - DNA, 20 bp, Artificial Sequence
  sgRNA coding sequence
tccagagagcacggtagacc

SEQ ID NO: 67 - DNA, 20 bp, Artificial Sequence
  sgRNA coding sequence
cggtagacctgggagccgct

SEQ ID NO: 68 - DNA, 20 bp, Artificial Sequence
  sgRNA coding sequence
gtggtcacccaggggctgcc

SEQ ID NO: 69 - DNA, 20 bp, Artificial Sequence
  sgRNA coding sequence
acccctgcgtggtcacccag

SEQ ID NO: 70 - DNA, 20 bp, Artificial Sequence
  sgRNA coding sequence
agacccctgcgtggtcaccc

SEQ ID NO: 71 - DNA, 20 bp, Artificial Sequence
  sgRNA coding sequence
tggcgggtcagacccctgcg

SEQ ID NO: 72 - DNA, 20 bp, Artificial Sequence
  sgRNA coding sequence
ggtggagaggacaaggctgg

SEQ ID NO: 73 - DNA, 20 bp, Artificial Sequence
  sgRNA coding sequence
cctggtggagaggacaaggc

SEQ ID NO: 74 - DNA, 20 bp, Artificial Sequence
  sgRNA coding sequence
catacctggtggagaggaca

SEQ ID NO: 75 - DNA, 20 bp, Artificial Sequence
  sgRNA coding sequence
agtgcacatacctggtggag

SEQ ID NO: 76 - DNA, 20 bp, Artificial Sequence
  sgRNA coding sequence
cgcgcagtgcacatacctgg

SEQ ID NO: 77 - DNA, 20 bp, Artificial Sequence
  sgRNA coding sequence
cgccagctcaccttcagttt

SEQ ID NO: 78 - DNA, 20 bp, Artificial Sequence
  sgRNA coding sequence
tcacgcgcagtgcacatacc

SEQ ID NO: 79 - DNA, 20 bp, Artificial Sequence
  sgRNA coding sequence
ccgccagctcaccttcagtt

SEQ ID NO: 80 - DNA, 20 bp, Artificial Sequence
  sgRNA coding sequence
acagtacagcaagggtctga

SEQ ID NO: 81 - DNA, 20 bp, Artificial Sequence
  sgRNA coding sequence
ctgctgcgccaaggcagtgg

SEQ ID NO: 82 - DNA, 20 bp, Artificial Sequence
  sgRNA coding sequence
tgctgcgccaaggcagtgga

SEQ ID NO: 83 - DNA, 20 bp, Artificial Sequence
  sgRNA coding sequence
aggcagtggagggtgagtcc

SEQ ID NO: 84 - DNA, 20 bp, Artificial Sequence
  sgRNA coding sequence
ggtgagtcctggcagcccct

SEQ ID NO: 85 - DNA, 20 bp, Artificial Sequence
  sgRNA coding sequence
agcccctgggtgaccacgca

SEQ ID NO: 86 - DNA, 20 bp, Artificial Sequence
  sgRNA coding sequence
gcccctgggtgaccacgcag

SEQ ID NO: 87 - DNA, 20 bp, Artificial Sequence
  sgRNA coding sequence
caaactgaaggtgagctggc

SEQ ID NO: 88 - DNA, 20 bp, Artificial Sequence
  sgRNA coding sequence
aaactgaaggtgagctggcg

SEQ ID NO: 89 - DNA, 20 bp, Artificial Sequence
  sgRNA coding sequence
aactgaaggtgagctggcgg

SEQ ID NO: 90 - DNA, 20 bp, Artificial Sequence
  sgRNA coding sequence
aaggtgagctggcgggggct

SEQ ID NO: 91 - DNA, 20 bp, Artificial Sequence
  sgRNA coding sequence
tctggcctcccagatccagg

SEQ ID NO: 92 - DNA, 20 bp, Artificial Sequence
  sgRNA coding sequence
gggatctggcgcccagcttc

SEQ ID NO: 93 - DNA, 20 bp, Artificial Sequence
  sgRNA coding sequence
aaccgggtgagacagggatc

SEQ ID NO: 94 - DNA, 20 bp, Artificial Sequence
  sgRNA coding sequence
aaggagaaccgggtgagaca

SEQ ID NO: 95 - DNA, 20 bp, Artificial Sequence
  sgRNA coding sequence
gaaggagaaccgggtgagac

SEQ ID NO: 96 - DNA, 20 bp, Artificial Sequence
  sgRNA coding sequence
cttgcgaggacctagggaag

SEQ ID NO: 97 - DNA, 20 bp, Artificial Sequence
  sgRNA coding sequence
ccttgcgaggacctagggaa

SEQ ID NO: 98 - DNA, 20 bp, Artificial Sequence
  sgRNA coding sequence
cccttgcgaggacctaggga

SEQ ID NO: 99 - DNA, 20 bp, Artificial Sequence
  sgRNA coding sequence
tcggcccttgcgaggaccta

SEQ ID NO: 100 - DNA, 20 bp, Artificial Sequence
  sgRNA coding sequence
ctcggcccttgcgaggacct

SEQ ID NO: 101 - DNA, 20 bp, Artificial Sequence
  sgRNA coding sequence
acagcccttaggggagtcca

SEQ ID NO: 102 - DNA, 20 bp, Artificial Sequence
  sgRNA coding sequence
cacagcccttaggggagtcc

SEQ ID NO: 103 - DNA, 20 bp, Artificial Sequence
  sgRNA coding sequence
cgtatcactcaccggagagc

SEQ ID NO: 104 - DNA, 20 bp, Artificial Sequence
  sgRNA coding sequence
gtcgaccacgtatcactcac

SEQ ID NO: 105 - DNA, 20 bp, Artificial Sequence
  sgRNA coding sequence
actggcagccttcaccctcc

SEQ ID NO: 106 - DNA, 20 bp, Artificial Sequence
  sgRNA coding sequence
agccttcaccctcctggatc

SEQ ID NO: 107 - DNA, 20 bp, Artificial Sequence
  sgRNA coding sequence
gccttcaccctcctggatct

SEQ ID NO: 108 - DNA, 20 bp, Artificial Sequence
  sgRNA coding sequence
ggatctgggaggccagaagc

SEQ ID NO: 109 - DNA, 20 bp, Artificial Sequence
  sgRNA coding sequence
gatctgggaggccagaagct

SEQ ID NO: 110 - DNA, 20 bp, Artificial Sequence
  sgRNA coding sequence
cgccagatccctgtctcacc

SEQ ID NO: 111 - DNA, 20 bp, Artificial Sequence
  sgRNA coding sequence
gctctccggtgagtgatacg

SEQ ID NO: 112 - DNA, 20 bp, Artificial Sequence
  sgRNA coding sequence
ggtgagtgatacgtggtcga

SEQ ID NO: 113 - DNA, 20 bp, Artificial Sequence
  sgRNA coding sequence
gtgagtgatacgtggtcgac

SEQ ID NO: 114 - DNA, 20 bp, Artificial Sequence
  sgRNA coding sequence
tgatacgtggtcgacgggtt

SEQ ID NO: 115 - DNA, 20 bp, Artificial Sequence
  sgRNA coding sequence
ggactgagctgtgtggctac

SEQ ID NO: 116 - DNA, 20 bp, Artificial Sequence
  sgRNA coding sequence
aggccattgtggagtggcac

SEQ ID NO: 117 - DNA, 20 bp, Artificial Sequence
  sgRNA coding sequence
gagcggacgtagggtctgtg

SEQ ID NO: 118 - DNA, 20 bp, Artificial Sequence
  sgRNA coding sequence
ggagcggacgtagggtctgt

SEQ ID NO: 119 - DNA, 20 bp, Artificial Sequence
  sgRNA coding sequence
tggagcggacgtagggtctg

SEQ ID NO: 120 - DNA, 20 bp, Artificial Sequence
  sgRNA coding sequence
ctcacttggtgtggctgtgc

SEQ ID NO: 121 - DNA, 20 bp, Artificial Sequence
  sgRNA coding sequence
actcacttggtgtggctgtg

SEQ ID NO: 122 - DNA, 20 bp, Artificial Sequence
  sgRNA coding sequence
ctgggggcctgatactcact

SEQ ID NO: 123 - DNA, 20 bp, Artificial Sequence
  sgRNA coding sequence
gttcctgtgccactccacaa

SEQ ID NO: 124 - DNA, 20 bp, Artificial Sequence
  sgRNA coding sequence
agccacaccaagtgagtatc

SEQ ID NO: 125 - DNA, 20 bp, Artificial Sequence
  sgRNA coding sequence
cactccctgtgggggtgaag

SEQ ID NO: 126 - DNA, 20 bp, Artificial Sequence
  sgRNA coding sequence
cggatgtccactccctgtgg

SEQ ID NO: 127 - DNA, 20 bp, Artificial Sequence
  sgRNA coding sequence
gcggatgtccactccctgtg

SEQ ID NO: 128 - DNA, 20 bp, Artificial Sequence
  sgRNA coding sequence
ggcggatgtccactccctgt

SEQ ID NO: 129 - DNA, 20 bp, Artificial Sequence
  sgRNA coding sequence
tggcggatgtccactccctg

SEQ ID NO: 130 - DNA, 20 bp, Artificial Sequence
  sgRNA coding sequence
tttcagaaataagtaataat

SEQ ID NO: 131 - DNA, 20 bp, Artificial Sequence
  sgRNA coding sequence
agtaataattggctatggtt

SEQ ID NO: 132 - DNA, 20 bp, Artificial Sequence
  sgRNA coding sequence
taataattggctatggttgg

SEQ ID NO: 133 - DNA, 20 bp, Artificial Sequence
  sgRNA coding sequence
tggctatggttgggggtaat

SEQ ID NO: 134 - DNA, 20 bp, Artificial Sequence
  sgRNA coding sequence
ggctatggttgggggtaatt

SEQ ID NO: 135 - DNA, 20 bp, Artificial Sequence
  sgRNA coding sequence
gttgggggtaattgggtcca

SEQ ID NO: 136 - DNA, 20 bp, Artificial Sequence
  sgRNA coding sequence
ggttgcctcttcacccccac

SEQ ID NO: 137 - DNA, 20 bp, Artificial Sequence
  sgRNA coding sequence
gttgcctcttcacccccaca

SEQ ID NO: 138 - DNA, 20 bp, Artificial Sequence
  sgRNA coding sequence
ctcttcacccccacagggag

SEQ ID NO: 139 - DNA, 20 bp, Artificial Sequence
  sgRNA coding sequence
agtggacatccgccataaca

SEQ ID NO: 140 - DNA, 20 bp, Artificial Sequence
  sgRNA coding sequence
ggatctgcaagtcagacctg

SEQ ID NO: 141 - DNA, 20 bp, Artificial Sequence
  sgRNA coding sequence
gaggatctgcaagtcagacc

SEQ ID NO: 142 - DNA, 20 bp, Artificial Sequence
  sgRNA coding sequence
gcttggtgccagcactagaa

SEQ ID NO: 143 - DNA, 20 bp, Artificial Sequence
  sgRNA coding sequence
gacccttcccaaagacctca

SEQ ID NO: 144 - DNA, 20 bp, Artificial Sequence
  sgRNA coding sequence
tgacccttcccaaagacctc

SEQ ID NO: 145 - DNA, 20 bp, Artificial Sequence
  sgRNA coding sequence
gctgttggtcaaggtgaggc

SEQ ID NO: 146 - DNA, 20 bp, Artificial Sequence
  sgRNA coding sequence
ctgttggtcaaggtgaggct

SEQ ID NO: 147 - DNA, 20 bp, Artificial Sequence
  sgRNA coding sequence
aggctgggccctgaggtctt

SEQ ID NO: 148 - DNA, 20 bp, Artificial Sequence
  sgRNA coding sequence
ggctgggccctgaggtcttt

SEQ ID NO: 149 - DNA, 20 bp, Artificial Sequence
  sgRNA coding sequence
gggccctgaggtctttggga

SEQ ID NO: 150 - DNA, 20 bp, Artificial Sequence
  sgRNA coding sequence
tctttgggaagggtcacccc

(19) United States
(12) Patent Application Publication
(10) Pub. No.: US 2021/0163936 A1
(43) Pub. Date: Jun. 3, 2021

(54) METHOD FOR SCREENING AND IDENTIFYING FUNCTIONAL LNCRNAS

(71) Applicants: PEKING UNIVERSITY, Beijing (CN); EDIGENE BIOTECHNOLOGY INC., Beijing (CN)

(72) Inventors: Wensheng WEI, Beijing (CN); Ying LIU, Beijing (CN); Zhongzheng CAO, Beijing (CN); Yinan WANG, Beijing (CN); Yu GUO, Beijing (CN); Pengfei YUAN, Beijing (CN)

(21) Appl. No.: 17/044,831

(22) PCT Filed: Apr. 2, 2018
(86) PCT No.: PCT/CN2018/081635

§ 371 Date: Oct. 1, 2020

(51) Int. Cl. C12N 15/113; C12N 9/22

(52) U.S. Cl. CPC C12N 15/113; C12N 9/22; C12N 2740/16043; C12N 2330/31; C12N 2310/20

(57) ABSTRACT

Provided is a high-throughput method for screening or identifying long non-coding RNAs by CRISPR system, which uses paired guide RNA targeting the genomic sequence within the region spanning −50 bp to +75 bp surrounding a splice donor site or a splice acceptor site of a long non-coding RNA.

### CROSS REFERENCE TO RELATED APPLICATIONS

This application is a National Phase application under 35 U.S.C. § 371 of International Application No. PCT/CN2018/081635, filed Apr. 2, 2018, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 794922002000SEQLIST.TXT, date recorded: Oct. 1, 2020, size: 29 KB).

### FIELD OF THE INVENTION

The invention is related to genetic perturbation of long non-coding RNAs (lncRNAs) by targeting splice sites in genome of a eukaryotic cell and thus screening and identifying functional lncRNAs.

### BACKGROUND OF THE INVENTION

As a powerful genome editing tool, the CRISPR-Cas9 system has been harnessed to identify gene functions through large-scale screens¹⁻⁴. The gene perturbation, even in genome-scale, is mostly through frameshift mutations generated within exons. Except for about 2% protein-coding genes in human genome, increasing evidence reveals that the rest massive number of the transcripts are non-coding RNAs⁵. Among them, lncRNAs>200 nucleotides represent a large subgroup without apparent protein-coding potential⁶⁻⁷. Previous studies indicated that the total number of human lncRNAs outstrips that of protein-coding genes and this number continues climbing⁸.

LncRNAs play critical roles in diverse cellular processes at transcriptional or post-transcriptional levels by cis- or trans-regulating gene expression⁹. Despite tens of thousands of loci on human genome that have been annotated to encode long noncoding RNAs (lncRNAs), their functions are largely unknown, essentially due to the lack of scalable loss-of-function method. Because lncRNAs are generally insensitive to reading frame alterations, it is difficult to apply CRISPR-Cas9 system in a conventional way to disrupt their expressions, not to mention in a large-scale. We have previously developed a deletion strategy through pgRNA library for the loss-of-function screen of lncRNAs⁹, but it is laborious to scale up. Although screens based on RNA interference^{10,11} or CRISPRi¹² were proved effective for the functional identifications of lncRNAs, RNAi method has potential off-target problems¹³, and both approaches are limited by the effectiveness of transcript knockdown. Therefore, there is a demand for an effective method to screen and identify functional long noncoding RNAs, and perturb noncoding RNA function in a large-scale fashion.

### SUMMARY OF THE INVENTION

This disclosure provides, inter alia, methods for studying the function of genomic regions, as well as methods for screening and identifying lncRNAs with function of regulation. These methods rely in part on a newly developed CRISPR/Cas system-based library screen provided herein.

In one aspect, the method of the invention exploits the ability of the CRISPR/Cas system to cleave specific genomic sequences around splice site of an lncRNA to introduce exon skipping or intron retention in the lncRNA and thus results in perturbation or elimination of the function of the lncRNA. The targeted genomic sites are specifically the genomic region around splice sites of a genomic gene coding for a long non-coding RNA (lncRNA), and the region is spanning −50-bp to +75-bp surrounding a SD site or SA site of the long non-coding RNA, more preferably, −30-bp to +30-bp, most preferably, −10-bp to +10-bp surrounding a SD site or SA site of the long non-coding RNA. The targeted sequences around splice site of a lncRNA are cleaved and mutated by cellular non-homologous end joining (NHEJ) machinery in the host cell, and such mutation results in exon skipping and/or intron retention and thus the function or activity of the lncRNA is eliminated substantially.

As is known in the art, CRISPR/Cas system nucleases require a guide RNA to cleave genomic DNA. These guide RNAs are composed of (1) a 19-21 nucleotide spacer sequence (guide sequence) of variable sequence that targets the CRISPR/Cas system nuclease to a genomic location in a sequence-specific manner, and (2) a hairpin sequence that is located between guide RNAs and allows the guide RNA to bind to the CRISPR/Cas system nuclease.

The methods provided herein involve introducing, into a host cell a CRISPR/Cas guide RNA construct comprising a guide sequence targeting a genomic sequence around a splice site of a long non-coding RNA and a hairpin sequence, operably linked to a promoter, expressing the guide RNA that targets the genomic sequence in the host cell. In one embodiment, the guide sequence targets a genomic sequence within the region spanning −50-bp to +75-bp surrounding a SD site or SA site of a long non-coding RNA, more preferably, −30-bp to +30-bp surrounding a SD site or SA site of a long non-coding RNA, most preferably, −10-bp to +10-bp surrounding a SD site or SA site of a long non-coding RNA.

In some instances, the method further comprises determining the functional profile of the long non-coding RNA. The expression of a genomic gene (coding gene or non-coding gene) or functional activity of its gene product (encoded protein) may be used as the readout of the regulatory function of the lncRNA. Alternatively, a coding sequence for a reporter gene may be inserted into the genome (e.g., in place of the native coding sequence) and the change of the expression or functional activity of its gene product may be used as a readout of the functional profile of the long non-coding RNA. In some instances, the coding sequence of a reporter gene is fused to the native coding sequence, and the readout is the mRNA or protein expression of the resultant fusion protein or the functional activity of the fusion protein.

In one aspect, the methods disclosed herein can be used to screen and identify lncRNAs involved in cellular processes other than transcription, including for example cell survival, cell division, cell metabolism, cell apoptosis, cell cycle, nucleosome assembly, signal transduction, multicellular organism development, immune reaction, cell adhesion, angiogenesis, etc. In some embodiments, the method can be used to identify lncRNAs that result in a change of a cellular process selecting from a group consisting of cell survival, cell division, cell metabolism, cell apoptosis, cell cycle, nucleosome assembly, signal transduction, multicellular organism development, immune reaction, cell adhesion and angiogenesis. In some embodiments, the method can be used to identify lncRNAs that result in a cellular phenotype change, for example, loss of function or gain of function. In some embodiments, the method can be used to identify lncRNAs that result in a decrease or increase of transcription of a coding gene and/or non-coding gene. The method may be used to identify the effect of one or more lncRNAs simultaneously or consequently, or individually or in some combinations.

As an example, a population of cells is transfected with a library of CRISPR/Cas guide RNAs with each encoding the variable sequence of a guide RNA targeting a genomic sequence around splice site of a lncRNA, and the guide RNAs are expressed in the cells, and in the presence of CRISPR/Cas the guide RNAs induce exon skipping and/or intron retention of the lncRNA. The RNA profile and transcriptome of each cell may be analyzed using techniques such as but not limited to single-cell RNA-seq technology. The analysis will reveal the consequence(s) of the genomic mutation on the RNA profile of the cell including the type and abundance of RNA molecules. The method can also be used to identify the nature (e.g., sequence) of the guide RNA that effected the exon skipping or intron retention. Thus, the effect of the exon skipping or intron retention can be observed on the entire cellular transcriptome at once by performing the experiment in a single cell.

Thus, provided herein is a CRISPR/Cas guide RNA construct comprising a guide sequence targeting a genomic sequence around a splice site of a long non-coding RNA and a hairpin sequence, operably linked to a promoter.

In some embodiments, the eukaryotic genome may be a human genome, and thus the CRISPR/Cas guide construct may be intended for use in human cells.

The guide sequence may be 19-21 nucleotides in length. The hairpin sequence may be less than 100 nucleotides, less than 80 nucleotides, less than 60 nucleotides, or about 40 nucleotides in length. In other embodiments, the hairpin sequence may be about 20-60 nucleotides in length. Once transcribed, the hairpin sequence can be bound to a CRISPR/Cas nuclease.

The CRISPR/Cas guide construct is DNA in nature and when transcribed produces a guide RNA.

Also provided is a population of cells comprising any of the preceding host cells. The population of host cells may be homogeneous or heterogeneous.

In some embodiments, the cell further comprises a CRISPR/Cas nuclease and/or a coding sequence for the CRISPR/Cas nuclease. In some embodiments, the cell further comprises a Cas9 nuclease and/or a coding sequence for Cas9 nuclease.

In some embodiments, the host cell has integrated into its genome a coding sequence for a reporter protein or a fusion protein comprising a reporter protein.

In some embodiments, the host cell is in a host cell population and each host cell independently comprises a unique guide RNA construct.

In some embodiments, each host cell expresses a unique functional guide RNA and under the involvement of the functional guide RNA, the host cell is mutated in a different genomic sequence relative to other host cells in the population.

Also provided is a high throughput method for screening or identifying long non-coding RNAs in a eukaryotic genome, comprising introducing into a population of host cells a library or a pool of CRISPR/Cas guide RNAs targeting genomic sequences around splice sites of the lncRNAs, wherein each host cell in the population of the host cells independently comprises a unique guide RNA, and expresses the unique guide RNA, and in the presence of a CRISPR/Cas nuclease, the targeted genomic sequences are cleaved and mutated, and thus resulting in exon skipping and/or intron retention of the lncRNAs.

In some embodiments, the high throughput method further comprises identifying the effect of lncRNAs on a change of cellular phenotype or expression of a coding gene or non-coding gene. In some embodiments, each host cell expresses a unique guide RNA and is mutated in a different genomic sequence relative to other host cells in the population. In some embodiments, the coding gene is exogenous or endogenous to the genome of the host cell. In some embodiments, the change of cellular phenotype includes loss of function or gain of function. In some embodiments, the change of expression of a coding gene or non-coding gene is decrease or increase of transcription of a coding gene or non-coding gene.

Also provided are lncRNAs screened or identified by the high throughput method disclosed herein. These lncRNAs include but not limit to XXbac-B135H6.15, RP11-848P1.5, AC005330.2, AP001062.9, AP005135.2, RP11-867G23.4, LINC01049, DGCR5, RP11-509A17.3, CTB-25J19.1, CTD-2517M22.17, CROCCP2, AC016629.8, CTC-490G23.4, RP11-117D22.1, AC067969.2, RP11-251M1.1, AC004471.9, AC004471.10, AC002472.11, RP11-429J17.7, RP11-56N19.5, TMEM191A, LL22NC03-102D1.18, LINC00410, LL22NC03-23C6.13, RP11-83J21.3, RP11-544A12.4, ANKRD62P1-PARP4P3, CTD-2031P19.5, XXbac-B444P24.8, RP11-464F9.21, TPTEP1, MIR17HG and BMS1P20, which can be used for regulating cell growth and proliferation.

Also provided is a method for perturbating or eliminating the function of a long non-coding RNA in a eukaryotic cell comprising introducing into the eukaryotic cell one or more CRISPR/Cas guide RNAs that target one or more polynucleotide sequences around one or more splice sites of the long non-coding RNA, whereby the one or more guide RNAs target the one or more polynucleotide sequences around the one or more splice sites of the long non-coding RNA and in the presence of Cas protein, the one or more polynucleotide sequences are cleaved, resulting in intron retention and/or exon skipping of the long non-coding RNA and thus perturbating or eliminating the function of the long non-coding RNA. In some embodiments, the guide RNA targets a polynucleotide sequence within the region spanning −50-bp to +75-bp surrounding a SD site or SA site of a long non-coding RNA. In some embodiments, the guide RNA targets a polynucleotide sequence within the region spanning −30-bp to +30-bp surrounding a SD site or SA site of a long non-coding RNA. In some embodiments, the guide RNA targets a polynucleotide sequence within the region spanning −10-bp to +10-bp surrounding a SD site or SA site of a long non-coding RNA. In some embodiments, the CRISPR/Cas nuclease is Cas9 or Cpf1. In some embodiments, the introducing into the cell is by a delivery system comprising viral particles, liposomes, electroporation, microinjection, conjugation, nanoparticles, exosomes, microvesicles, or a gene-gun, preferably, by a delivery system comprising lentiviral particles.

### BRIEF DESCRIPTION OF THE DRAWINGS

FIG. 1. a, Genomic sequence features and base specificity of splice sites in human. The y axis indicates the probability of bases at each locus. b, Schematic of intron retention or exon skipping induced by sgRNAs targeting around splicing donor (SD) or splicing acceptor (SA) site.

FIG. 2. The figure shows the correlations between replicates in sgRNA library screening on essential ribosomal genes. Scatter plots of normalized sgRNA read counts of the splicing-targeting libraries including Day-0 control samples (Ctrl) and Day-15 experimental samples (Exp) in HeLa cell line (a) and Huh7.5 cell line (b). The Spearman correlation coefficients (Spearman corr.) between two replicates of each sample are also reported.

FIG. 3. The figure manifests deep sequencing analysis of CRISPR screen of the sgRNA library targeting ribosomal genes in HeLa and Huh7.5 cell lines. The sgRNA saturation mutagenesis library was designed to target −50-bp to +75-bp regions surrounding 5′ SD sites and −75-bp to +50-bp regions surrounding 3′ SA sites of 79 ribosomal genes. The pooled plasmid library was lentivirally transduced into HeLa and Huh7.5 cells expressing Cas9 protein, respectively. The dropouts of all sgRNAs at every indicated locus were calculated as log₂(Exp:Ctrl) of the normalized read counts and the black bar represents the mean fold change of all sgRNAs at each locus. The dotted lines indicated the positions of splice sites.

FIG. 4. The figure shows the identification of sgRNA-targeting regions for generating splice site disruption. a, Normalization of high-efficient sgRNAs at every locus in HeLa and Huh7.5 cell lines. Data were calculated by dividing the number of sgRNA with more than 4-fold dropout by the total number of designed sgRNAs at the indicated locus. b, Comparison of high-efficient sgRNAs targeting introns, 5′ SD sites and exons in HeLa and Huh7.5 cell lines. Each bar represents the percentage of sgRNAs with more than 2-fold or 4-fold dropout in different regions. Data are presented as the mean±s.e.m. c, Comparison of high-efficient sgRNAs targeting introns, 3′ SA sites and exons in HeLa and Huh7.5 cell lines. Data are presented as the mean±s.e.m.

FIG. 5. The figure illustrates the construction of the CRISPR system and the genome-scale screen to identify essential lncRNAs for cell growth and proliferation. a, Construction of the CRISPR system. b, The workflow of splicing-targeting sgRNA library construction, screening and data analysis. c, Scatter plot of sgRNA fold change between two independent replicates. d, The log₂(fold change) distribution of non-targeting sgRNAs, sgRNAs targeting essential genes and lncRNAs. The fold changes of each group were compared with non-targeting sgRNAs by student t-test. ***P<0.001. e, Screen scores of negatively selected lncRNAs by splicing-targeting CRISPR screening. For each lncRNA, the fold changes of all targeting sgRNAs were compared with negative control sgRNAs by Wilcox test and the generated P value was further corrected by the null distribution of negative control genes, which were obtained by randomly sampling negative control sgRNAs. The screen score was calculated from the mean fold change and corrected P value (see Methods). The top 10 lncRNA hits and negatively selected essential genes are labeled respectively.

FIG. 6. The figure shows the validation of the function of candidate lncRNAs. a-c, Effects of indicated sgRNAs on cell proliferation in K562 and GM12878 cells, which include three kinds of control sgRNAs, non-targeting sgRNA, sgRNA targeting AAVS1 locus, sgRNA targeting splice site of RPL18—an essential gene for cell growth (a), and two negatively selected lncRNAs (b, c). Each lentivirus of the sgRNA expression vector harboring a CMV promoter-driven EGFP marker was respectively transduced into K562 and GM12878 cells. The percentage of EGFP positive cells was measured every 3 days by FACS, indicating the fraction of sgRNA-infected cells. The first FACS analysis started at 3 days post infection (labeled as Day 0), then the pooled cells were passaged for 12 days. Cell proliferation of each sample was determined by dividing the percentages of EGFP positive cells at indicated time points by that at Day 0. Data are presented as the mean and standard derivation of three biological replicates. Asterisk (*) represents P value compared with sgRNA targeting AAVS1 at the assay end point (Day 12), calculated using Student's t-test and adjusted using Benjamini-Hochberg method. *P<0.05; **P<0.01; ***P<0.001; ****P<0.0001; NS, not significant. d, Cell proliferation of 35 top candidate lncRNAs in K562 cells compared with that in GM12878 cells by splicing-targeting strategy. The 35 top candidate lncRNAs are XXbac-B135H6.15, RP11-848P1.5, AC005330.2, AP001062.9, AP005135.2, RP11-867G23.4, LINC01049, DGCR5, RP11-509A17.3, CTB-25J19.1, CTD-2517M22.17, CROCCP2, AC016629.8, CTC-490G23.4, RP11-117D22.1, AC067969.2, RP11-251M1.1, AC004471.9, AC004471.10, AC002472.11, RP11-429J17.7, RP11-56N19.5, TMEM191A, LL22NC03-102D1.18, LINC00410, LL22NC03-23C6.13, RP11-83J21.3, RP11-544A12.4, ANKRD62P1-PARP4P3, CTD-2031P19.5, XXbac-B444P24.8, RP11-464F9.21, TPTEP1, MIR17HG, BMS1P20. The threshold was set at 80%, the normalized percentage of sgRNA-infected cells at Day 12. Light grey dots indicate lncRNAs essential only in K562 cells and heavy grey dots indicate those exhibiting growth phenotypes in both K562 and GM12878 cells. e, Effects of large-fragment deletions of lncRNA XXbac-B135H6.15 on cell proliferation in K562 cells. 4 pairs of gRNAs were designed to delete the promoter and the first exon. The pgRNAs also expressed from the backbone containing EGFP marker and the cell proliferation assay was performed as in FIG. 3 (a-c). Data are presented as the mean value and standard derivation of three biological replicates. Asterisks represent P values compared with AAVS1_p1 at Day 15, which were calculated using Student's t-test and adjusted using Benjamini-Hochberg method. *P<0.05; **P<0.01; ***P<0.001; ****P<0.0001; NS, not significant. f, The correlations of knockout effects on top lncRNA candidates between splicing-targeting and pgRNA-mediated deletion methods.

FIG. 7-FIG. 12. These figures provide validation evidence for top-ranking lncRNAs through splicing-targeting strategy.

FIG. 13. This figure provides the validation of candidate lncRNAs through large-fragment deletion. a, Cell proliferation assay performed by large-fragment deletions of the AAVS1 locus and essential genes RPL19, RPL23A in K562 cells. 2 pairs of gRNAs were designed for AAVS1 locus, and one pair was designed for each essential gene to delete the promoter and the first exon. The design rule of pgRNAs and the method for determining growth effect were the same as described in FIG. 3e and for the remaining figure. Data are presented as the mean value and standard derivation of three biological replicates. Asterisks represent P values compared with AAVS1_p1 at Day 15, which were calculated using Student's t-test and adjusted using Benjamini-Hochberg method. *P<0.05; **P<0.01; ***P<0.001; ****P<0.0001; NS, not significant. b, Effects of large-fragment deletions on cell growth of 5 candidate lncRNAs which were also validated by splicing-targeting strategy.

FIG. 14. The figure provides validation of candidate lncRNAs through large-fragment deletion, wherein 6 candidate lncRNAs were not validated by splicing-targeting strategy in K562 cells.

FIG. 15. The figure demonstrates the functional dissection of lncRNAs MIR17HG and BMS1P20 in K562 and GM12878 cell lines. a, Expression patterns of the top 500 genes showing the highest variance across MIR17HG- and BMS1P20-KO (knockout) cells and their corresponding controls. b, The expression levels of the top 100 essential lncRNA candidates in K562 and GM12878 cells. c, The expression levels of down-regulated essential genes in MIR17HG- and BMS1P20-KO cells compared with the wild-type K562 cells. d, Veen diagram of the essential genes showing down-regulation between MIR17HG- and BMS1P20-KO K562 cells. e, Volcano plots for differential expression following infection of splicing-targeting sgRNAs of BMS1P20 in K562 cells compared with in GM12878 cells. Black and grey dots represent all genes and differentially expressed genes, respectively. f, The Gene Ontology (GO) terms and KEGG annotations of genes that were down-regulated (top) and up-regulated (bottom) in K562 cells.

FIG. 16. The figure illustrates RNA-seq profiling of lncRNA knockouts of MIR17HG and BMS1P20 in K562 and GM12878 cells. a, Paired scatter plot of the gene expression levels across MIR17HG-KO (knockout), BMS1P20-KO and wild-type K562 cells. b, Paired scatter plot of the gene expression levels across MIR17HG knockouts, BMS1P20 knockouts and wild-type GM12878 cells. c, The Gene Ontology and KEGG annotations of conserved essential genes showing down-regulation after infecting splicing-targeting sgRNAs of MIR17HG and BMS1P20 in K562 cells. d, Volcano plots for differential expression between BMS1P20-KO and wild-type K562 cells. e, Volcano plots for differential expression between BMS1P20-KO and wild-type GM12878 cells.

### DETAILED DESCRIPTION OF THE INVENTION

### Definition

The present invention will be described with respect to particular embodiments and with reference to certain drawings but the invention is not limited thereto but only by the claims. Any reference signs in the claims shall not be construed as limiting the scope. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non limiting examples of polynucleotides: coding or non-coding regions of a gene or gene fragment, loci (locus), exons, introns, messenger RNA (mRNA), long non-coding RNA (lncRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non nucleotide components. A polynucleotide may be further modified after polymerization, such as by conjugation with a labeling component.

In aspects of the invention the terms “chimeric RNA”, “chimeric guide RNA”, “guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PGR 2: A PRACTICAL APPROACH (M. J. MacPherson, B. D. Hames and GR. Taylor eds. (1995)), Harlow and Lane, eds. (1988) ANTIBODIES, A LABORATORY MANUAL, and ANIMAL CELL CULTURE (R. L. Freshney, ed. (1987))¹⁴⁻¹⁸.

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells, yeast cells, or mammalian cells. Suitable host cells are also recited in Goeddel, GENE EXPRESSION TECHNOLOGY: METHODS IN ENZYMOLOGY 185, Academic Press, San Diego, Calif. (1990)¹⁹. Alternatively, the recombinant expression vector can be transcribed and translated in vitro, for example using T7 promoter regulatory sequences and T7 polymerase.

In some embodiments, a vector is capable of driving expression of one or more sequences in mammalian cells using a mammalian expression vector. Examples of mammalian expression vectors include pCDM8²⁰ and pMT2PC²¹. When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989¹⁴.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system.

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 23, 26, 29, 32, 35, 38, 41, 44, 47, 50, 53, 56, 59, 62, 65, 70, 75, 80, 85 or more nucleotides of a wild-type tracr sequence), may also form part, of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

In some embodiments, one or more vectors driving expression of one or more elements of a CRISPR system are introduced into a host cell such that expression of the elements of the CRISPR system directs formation of a CRISPR complex at one or more target sites. In another embodiment, the host cell is engineered to stably express Cas9 and/or OCT1.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wimsch algorithm, algorithms based on the Burrows-Wheeler Transform (e.g. the Burrows Wheeler Aligner), ClustalW, Clustai X, BLAT, Novoalign (Novocraft Technologies, ELAND ((Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 or more nucleotides in length. In some embodiments, a guide sequence is less than about 75, 70, 65, 60, 55, 50, 45, 40, 35, 30, 25, 20, 15, 12, or fewer nucleotides in length. The ability of a guide sequence to direct sequence-specific binding of a CR1SPR complex to a target sequence may be assessed by any suitable assay. For example, the components of a CRJSPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, RNA cleavage activity and nucleic acid binding activity.

In some aspects, the invention provides methods comprising delivering one or more polynucleotides, such as or one or more constructs including vectors as described herein, one or more transcripts thereof, and/or one or proteins transcribed therefrom, to a host cell. The invention serves as a basic platform for enabling targeted modification of DNA-based genomes. It can interface with many delivery systems, including but not limited to viral, liposome, electroporation, microinjection and conjugation. In some aspects, the invention further provides cells produced by such methods, and organisms (such as animals, plants, or fungi) comprising or produced from such cells. In some embodiments, a CRISPR enzyme in combination with (and optionally complexed with) a guide sequence is delivered to a cell. Conventional viral and non-viral based gene transfer methods can be used to introduce nucleic acids into mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes for delivery to the cell.

Methods of non-viral delivery of nucleic acids include lipofection, nucleofection, microinjection, biolistics, virosomes, liposomes, immunoliposomes, polycation or lipid:nucleic acid conjugates, naked DNA and artificial virions.

The use of RNA or DNA viral based systems for the delivery of nucleic acids has high efficiency advantage in targeting a virus to specific cells in the body and trafficking the viral payload to the nucleus.

In preferred embodiments, targets of the present invention include long noncoding RNAs (lncRNAs), which represent a class of long transcribed RNA molecules, for example, the RNA molecules longer than 200 nucleotides. Their size distinguishes lncRNAs from small regulatory RNAs such as microRNAs (miRNAs), short interfering RNAs (siRNAs), Piwi-interacting RNAs (piRNAs), small nucleolar RNAs (snoRNAs), short hairpin RNA (shRNA), and other short RNAs. LncRNAs may function by binding to DNA or RNA in a sequence specific manner or by binding to proteins. In contrast to miRNAs, lncRNAs appear not to operate by a common mode of action but can regulate gene expression and protein synthesis in a number of ways.

lncRNAs can be classified into the following locus biotypes based on their location with respect to protein-coding genes. Intergenic lncRNA, which are transcribed inter genetically from both strands; Intronic lncRNA, which are entirely transcribed from introns of protein-coding genes; Sense lncRNA, which are transcribed from the sense strand of protein-coding genes and contain exons from protein-coding genes that overlap with part of protein-coding genes or cover the entire sequence of a protein-coding gene through an intron; and Antisense lncRNA, which are transcribed from the antisense strand of the protein-coding genes that overlap with exonic or intronic regions, or cover the entire protein-coding sequence through an intron. Recent research in human transcriptome analysis shows that protein-coding sequences only account for a small portion of the genome transcripts. The majority of the human genome transcripts are non-coding RNAs.

The term “lncRNA” refers broadly to the targets of the present invention and include the “lncRNA gene”, as well as the resultant “lncRNA transcript.”

As used herein, the term “exon” indicates any part of a gene that will encode a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

An “intron” is any nucleotide sequence within a gene that is removed by RNA splicing during maturation of the final RNA product. The term intron refers to both the DNA sequence within a gene and the corresponding sequence in RNA transcripts. Sequences that are joined together in the final mature RNA after RNA splicing. Introns are found in the genes of most organisms and many viruses, and can be located in a wide range of genes, including those that generate proteins, ribosomal RNA (rRNA), long non-coding RNA (lncRNA) and transfer RNA (tRNA). When proteins are generated from intron-containing genes, RNA splicing takes place as part of the RNA processing pathway that follows transcription and precedes translation.

The term “splicing” as used herein means editing of a nascent precursor RNA into mature RNA, for example, editing nascent precursor messenger RNA (pre-mRNA) transcript into a mature messenger RNA (mRNA). For many eukaryotic introns, splicing is carried out in a series of reactions which are catalyzed by the spliceosome, a complex of small nuclear ribonucleoproteins (snRNPs). Spliceosomal introns often reside within the sequence of eukaryotic protein-coding genes. Within the intron, a donor site (5′ end of the intron), a branch site (near the 3′ end of the intron) and an acceptor site (3′ end of the intron) are required for splicing. The splice donor (SD) site includes an almost invariant sequence GT at the 5′ end of the intron, within a larger, less highly conserved region. The splice acceptor (SA) site at the 3′ end of the intron terminates the intron with an almost invariant AG sequence. Upstream (5′-ward) from the AG there is a region high in pyrimidines (C and T), or polypyrimidine tract. Further upstream from the polypyrimidine tract is the branchpoint, which includes an adenine nucleotide involved in lariat formation^{22, 23}.

Nuclear pre-mRNA introns are characterized by specific intron sequences located at the boundaries between introns and exons. These sequences are recognized by spliceosomal RNA molecules when the splicing reactions are initiated. The major spliceosome splices introns containing GT at the 5′ splice site and AG at the 3′ splice site, and this type of splicing is termed canonical splicing or termed the lariat pathway, which accounts for more than 99% of splicing. By contrast, when the intronic flanking sequences do not follow the GT-AG rule, noncanonical splicing is said to occur which accounts for less than 1% of splicing²⁴.

Our bioinformatics analysis using Weblogo3 tools shows that about 99% intronic regions in human genome are flanked by GT at the 5′ sites and AG at the 3′ sites. These intronic regions are applicable for coding genes and noncoding RNAs.

Exon skipping is a form of RNA splicing which causes “skipping” of one or more exons over the resultant RNA, while “intron retention” is a form of RNA splicing in which an intron is simply retained in the resultant RNA after splicing.

Splicing is regulated by trans-acting proteins (repressors and activators) and corresponding cis-acting regulatory sites (silencers and enhancers) on the pre-mRNA. However, as part of the complexity of alternative splicing, it is noted that the effects of a splicing factor are frequently position-dependent. That is, a splicing factor that serves as a splicing activator when bound to an intronic enhancer element may serve as a repressor when bound to its splicing element in the context of an exon, and vice versa²⁵. The secondary structure of the pre-mRNA transcript also plays a role in regulating splicing, such as by bringing together splicing elements or by masking a sequence that would otherwise serve as a binding element for a splicing factor²⁶. Together, these elements form a “splicing code” that governs how splicing will occur under different cellular conditions²⁷.

Modification of a Gene in a Eukaryotic Cell

The present method is related to effectively delivering an sgRNA targeting splice site to generate exon skipping and/or intron retention to perturb a gene, for example a coding gene or noncoding gene. For a gene coding for lncRNA, the method can effectively affect the function of the lncRNA.

To assess the power of splicing-targeting in CRISPR screen, we designed a saturation library targeting splice sites of 79 ribosomal genes, most of which were essential for cellular growth in various cell lines. This library contained 5,788 sgRNAs whose cutting sites are within □50-bp to +75-bp surrounding every 5′ SD site and □75-bp to +50-bp surrounding every 3′ SA site of these 79 genes. It became evident that sgRNAs affecting splice sites outperformed those targeting only exonic regions, and the closer the distances from sgRNAs' cutting sites to splice sites, the better their effects on gene disruption, with peak points slightly towards the exons for both SD and SA cases.

CRISPR/Cas9 Mechanism of Action and Library Screening Rationale

The method of the present invention utilizes the CRISPR/Cas system. Cas9 is a nuclease from the microbial type II CRISPR (clustered regularly interspaced short palindromic repeats) system, which has been shown to cleave DNA when paired with a single-guide RNA (gRNA). The gRNA contains a 17-21 bp sequence that directs Cas9 to complementary regions in the genome, thus enabling site-specific creation of double-strand breaks (DSBs) that are repaired in an error-prone fashion by cellular non-homologous end joining (NHEJ) machinery. Cas9 primarily cleaves genomic sites at which the gRNA sequence is followed by a PAM sequence (-NGG). NHEJ-mediated repair of Cas9-induced DSBs induces a wide range of mutations initiated at the cleavage site which are typically small (<10 bp) insertion/deletions (indels) but can include larger (>100 bp) indels and altered individual bases.

The splicing-targeting method of the present invention can be used to screen a plurality (e.g., thousands) of sequences in the genome, thereby elucidating the function of such sequences. In some embodiments, the splicing-targeting method of the present invention involves in a high-throughput screen for long non-coding RNAs by using CRISPR/Cas9 system to identify genes required for survival, proliferation or drug resistance and so on. In the screen, gRNAs targeting tens of thousands of splicing sites within genes of interest are delivered, for example, by lentiviral vectors, as a pool, into target cells along with Cas9. By identifying gRNAs that are enriched or depleted in the cells after selection for the desired phenotype, genes that are required for this phenotype can be systematically identified.

In the above high-throughput CRISPR/Cas9-based approach, the gRNA libraries can be cloned into lentiviral vectors. In this situation, it is necessary to lower the multiplicity of infection (MOI) to limit the number of guide RNAs in a single cell, typically having only a single guide RNA per cell. It is random which gRNA is integrated in each cell, allowing a pooled screen in which each cell expresses only one gRNA. Of note, the genomic gRNA-based high-throughput screen targeting splice sites of the present invention could also be applied to other CRISPR-based high-throughput screens for coding genes and regulatory genes.

Guide RNAs

As is known in the art, CRISPR/Cas system nucleases require a guide RNA to cleave genomic DNA. These guide RNAs are composed of (1) a 19-21 nucleotide spacer (guide) of variable sequence (guide sequence) that targets the CRISPR/Cas system nuclease to a genomic location in a sequence-specific manner, and (2) an invariant hairpin sequence that is constant between guide RNAs and allows the guide RNA to bind to the CRISPR/Cas system nuclease. In the presence a CRISPR/Cas nuclease, the guide RNA triggers a CRISPR/Cas-based genomic cleavage event in a cell.

A guide sequence is selected or designed based on the contemplated target sequence. In some embodiments, the target sequence is a sequence around splice site, for example, −50-bp to +75-bp surrounding SD site, preferred the −30-bp to +30-bp region surrounding SD site, and most preferred the −10-bp to +10-bp region surrounding SD site; −50-bp to +75-bp region surrounding SA site, preferred the −30-bp to +30-bp region surrounding SA site, and most preferred the −10-bp to +10-bp region surrounding SA site of a gene coding for a lncRNA within a genome of a cell. Exemplary target sequences include those that are unique in the target genome.

For example, for the S. pyogenes Cas9, a unique target sequence in a genome may include a Cas9 target site of the form M₈N₁₂XGG where N₁₂XGG (N is A, G, T, or C; and X can be anything) has a single occurrence in the genome. A unique target sequence in a genome may include an S. pyogenes Cas9 target site of the form M₉N₁₁AGG where N₁₁XGG (N is A, G, T, or C; and X can be anything) has a single occurrence in the genome.

For the S. thermophilus CRISPR1 Cas9, a unique target sequence in a genome may include a Cas9 target site of the form M₈N₁₂XXAGAAW where N₁₂XXAGAAW (N is A, G, T, or C; X can be anything; and W is A or T) has a single occurrence in the genome. A unique target sequence in a genome may include an S. thermophilus CRISPR1 Cas9 target site of the form M₉N₁₁XXAGAAW where N₁₁XXAGAAW (N is A, G, T, or C; X can be anything; and W is A or T) has a single occurrence in the genome.

For the S. pyogenes Cas9, a unique target sequence in a genome may include a Cas9 target site of the form M₈N₁₂XGGXG where N₁₂XGGXG (N is A, G, T, or C; and X can be anything) has a single occurrence in the genome. A unique target sequence in a genome may include an S. pyogenes Cas9 target site of the form M₉N₁₁XGGXG where N₁₁XGGXG (N is A, G, T, or C; and X can be anything) has a single occurrence in the genome. In each of these sequences “M” may be A, G, T, or C, and need not be considered in identifying a sequence as unique.

It is to be understood that any hairpin sequence can be used provided it can be recognized and bound by a CRISPR/Cas nuclease.

Guide RNA Constructs

In certain embodiments, the present invention is related to a guide RNA construct. The guide RNA construct may comprise (1) a guide sequence and (2) a guide RNA hairpin sequence, and optionally (3) a promoter sequence capable of initiating guide RNA transcription. A non-limiting example of a guide RNA hairpin sequence is the FE hairpin sequence described in Chen et al. Cell. 2013 Dec. 19; 155(7): 1479-91. An example of a promoter is the human U6 promoter.

In certain embodiments, the present invention is related to CRISPR/Cas guide construct comprising (1) a guide sequence and (2) a guide RNA hairpin sequence, and optionally (3) a promoter sequence capable of initiating guide RNA transcription, wherein the guide sequence targeting a sequence around splice site in a eukaryotic genome, for example, the guide sequence targets the −50-bp to +75-bp region surrounding SD site or SA site, preferred the −30-bp to +30-bp region surrounding SD site or SA site, and most preferred the −10-bp to +10-bp region surrounding SD site or SA site of a gene coding for lncRNA. In certain embodiments, the guide sequence targets splice site of a gene coding for a long non-coding RNA in the eukaryotic genome to induce exon skipping and/or intron retention, and thus disrupting the long non-coding RNA. In certain embodiments, the eukaryotic genome is a human genome. In certain embodiments, the guide sequence is 19-21 nucleotides in length. In certain embodiments, the hairpin sequence is about 40 nucleotides in length and once transcribed can be bound to a CRISPR/Cas nuclease.

CRISPR/Cas System Nucleases

In some embodiments, the CRISPR/Cas nuclease is a type II CRISPR/Cas nuclease. In some embodiments, the CRISPR/Cas nuclease is Cas9 nuclease. In some embodiments, the Cas9 nuclease is S. pneumoniae, S. pyogenes, or S. thermophilus Cas9, and may include mutated Cas9 derived from these organisms. The nuclease may be a functionally equivalent variant of Cas9. In some embodiments, the CRISPR/Cas nuclease is codon-optimized for expression in a eukaryotic cell. In some embodiments, the CRISPR/Cas nuclease directs cleavage of one or two strands at the location of the target sequence. The CRISPR/Cas system nucleases include but are not limited to Cas9 and Cpf1.

Reporter Genes and Proteins, and Readouts

The reporter gene may be integrated into a cell using a CRISPR/Cas mechanism, in some embodiments. For example, an expression vector, such as a plasmid, may be used that comprises a promoter (e.g., U6 promoter), a guide RNA hairpin sequence, and a guide sequence that targets the desired genomic locus where the reporter construct is to be integrated. Such an expression vector may be generated by cloning the guide sequence into an expression construct comprising the remaining elements. A DNA fragment comprising the coding sequence for the reporter protein can be generated and subsequently modified to include homology arms that flank the coding sequence of the reporter protein. The guide RNA expression vector, the amplified DNA fragments comprising the reporter protein coding sequence, and a CRISPR/Cas nuclease (or an expression vector encoding the nuclease) are introduced into the host cell (e.g., via electroporation). The expression vectors may further comprise additionally selection markers such as antibiotic resistance markers to enrich for cells successfully transfected with the expression vectors. Cells that express the reporter protein can be further selected.

Reporter genes are used for identifying potentially transfected cells and for evaluating the functionality of regulatory sequences. In general, a reporter gene is a gene that is not endogenous or native to the host cells and that encodes a protein that can be readily assayed. Reporter genes that encode for easily assayable proteins are known in the art, including but not limited to, green fluorescent protein (GFP), glutathione-S-transferase (GST), horseradish peroxidase (HRP), chloramphenicol acetyltransferase (CAT) beta-galactosidase, beta-glucuronidase, luciferase, HcRed, DsRed, cyan fluorescent protein (CFP), yellow fluorescent protein (YFP), and autofluorescent proteins including blue fluorescent protein (BFP), cell surface markers, antibiotic resistance genes such as neo, and the like.

Expression Vectors

The term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Expression vectors in recombinant DNA techniques often take the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Host Cells

Virtually any eukaryotic cell type can be used as a host cell provided it can be cultured in vitro and modified as described herein. Preferably, the host cells are pre-established cell lines. The cells and cell lines may be human cells or cell lines, or they may be non-human, mammalian cells or cell lines.

### Example

### Materials and Methods

### 1. Cells and Reagents

The HeLa cell line was from Z. Jiang's laboratory (Peking University) and cultured in Dulbecco's modified Eagle's medium (DMEM, Gibco C11995500BT). Huh 7.5 cell line from S. Cohen's laboratory (Stanford University School of Medicine) was cultured in DMEM (Gibco) supplemented with 1% MEM non-essential amino acids (NEAA, Gibco 1140-050). K562 cell from H. Wu's laboratory (Peking University) and GM12878 cell from Coriell Cell Repositories were cultured in RPMI1640 medium (Gibco 11875-093). All cells were supplemented with 10% fetal bovine serum (FBS, CellMax BL102-02) with 1% penicillin/streptomycin, cultured with 5% CO₂ in 37° C.

### 2. Reverse Transcription PCR (RT-PCR) for Testing Intron Retention or Exon Skipping

The sgRNAs were cloned into a lentiviral expression vector carrying a CMV promoter-driven mCherry marker, then transduced into HeLaoc cells¹⁻⁴ through viral infection at an MOI of <1. 72 hrs post infection, the mCherry positive cells were FACS-sorted and the total RNA of each sample was extracted using RNAprep pure Cell/Bacteria Kit (TIANGEN DP430). The cDNAs were synthesized from 2 μg of total RNA using Quantscript RT Kit (TIANGEN KR103-04), and the RT-PCR reactions were performed with TransTaq HiFi DNA Polymerase (TransGen AP131-13).

Sequences of sgRNAs targeting RPL18 or RPL11 gene:
sgRNA1_{RPL18}:
(SEQ ID No: 1)
5′-GGACCAGCCACTCACCATCC
sgRNA2_{RPL18}:
(SEQ ID No: 2)
5′-AGCTTCATCTTCCGGATCTT
sgRNA3_{RPL11}:
(SEQ ID No: 3)
5′-TCCTTGTGACTACTCACCTT
sgRNA4_{RPL11}:
(SEQ ID No: 4)
5′-AACTCATACTCCCGCACCTG
Primers used for RT-PCR:
1F:
(SEQ ID No: 5)
5′-CTGGGTCTTGTCTGTCTGGAA;
1R:
(SEQ ID No: 6)
5′-CTGGTGTTTACATTCAGCCCC;
2F:
(SEQ ID No: 7)
5′-GGCCAGAAGAACCAACTCCA;
2R:
(SEQ ID No: 8)
5′-GACAGTGCCACAGCCCTTAG;
3F:
(SEQ ID No: 9)
5′-TCAAGATGGCGTGTGGGATT;
3R:
(SEQ ID No: 10)
5′-GACCAGCAAATGGTGAAGCC;
4F:
(SEQ ID No: 11)
5′-GATCCTTTGGCATCCGGAGA;
4R:
(SEQ ID No: 12)
5′-GCTGATTCTGTGTTTGGCCC.

3. Construction and Screening of Splicing-Targeting sgRNA Library on Essential Ribosomal Genes

The annotations of 79 ribosomal genes were retrieved from NCBI. We scanned all potential sgRNAs targeting −50-bp to +75-bp surrounding every 5′ SD site and −75-bp to +50-bp surrounding every 3′ SA site of these 79 genes including RPL10,RPL10A,RPL11,RPL12,RPL13,RPL13A,RPL14,RPL15,RPL17,RPL18,RPL1 8A,RPL19,RPL21,RPL22,RPL22L1,RPL23,RPL23A,RPL24,RPL26,RPL26L1,RPL2 7,RPL27A,RPL28,RPL29,RPL3,RPL30,RPL31,RPL32,RPL34,RPL35,RPL35A,RPL 36,RPL36A,RPL36AL,RPL37,RPL37A,RPL38,RPL39,RPL39L,RPL3L,RPL4,RPL4 1,RPL5,RPL6,RPL7,RPL7A,RPL7L1,RPL8,RPL9,RPS10,RPS11,RPS12,RPS13,RPS 14,RPS15,RPS15A,RPS16,RPS19,RPS2,RPS20,RPS21,RPS23,RPS24,RPS25,RPS26,RPS27,RPS27A,RPS27L,RPS28,RPS29,RPS3,RPS3A,RPS4X,RPS4Y1,RPS4Y2,RP S5,RPS6,RPS7,RPS8. We ensured that all sgRNAs had at least 2 mismatches to any other loci of the human genome. In order to exhibit the natural cleavage efficacy of sgRNAs in the library, the GC content was not considered in the design. Total of 5,788 sgRNAs targeting 79 ribosomal genes were synthesized using CustmoArray 12K array chip (CustmoArray, Inc.). Here taking the RPL18 gene among the 79 ribosomal genes as an example to illustrate the design of the sgRNAs.

| | | | | | Splice | Distance | |
|---|---|---|---|---|---|---|---|
| | | | | | site of | to | |
| | | | | SEQ | intron | splice | |
| | | | | ID | for | site | |
| sgRNA_ID | Gene_symbol | Gene_ID | sgRNA_sequence | NO. | targeting | (bp) | Location |
| in785887_a_106 | RPL18 | 6141 | AAAACCACGGCGGATGGCAG | 13 | 5′ end | 41 | intron |
| in785887_a_112 | RPL18 | 6141 | TAGCCCAAAACCACGGCGGA | 14 | 5′ end | 47 | intron |
| in785887_a_116 | RPL18 | 6141 | CCCCTAGCCCAAAACCACGG | 15 | 5′ end | 51 | intron |
| in785887_a_119 | RPL18 | 6141 | GTGCCCCTAGCCCAAAACCA | 16 | 5′ end | 54 | intron |
| in785887_a_1721 | RPL18 | 6141 | CCCGCAGCCTTCCAGTGAAG | 17 | 3′ end | 61 | intron |
| in785887_a_1722 | RPL18 | 6141 | CCCCGCAGCCTTCCAGTGAA | 18 | 3′ end | 60 | intron |
| in785887_a_1723 | RPL18 | 6141 | CCCCCGCAGCCTTCCAGTGA | 19 | 3′ end | 59 | intron |
| in785887_a_1775 | RPL18 | 6141 | ACCTGTATAACTGGAGGGAC | 20 | 3′ end | 7 | intron |
| in785887_a_1780 | RPL18 | 6141 | CAGAAACCTGTATAACTGGA | 21 | 3′ end | 2 | intron |
| in785887_a_1781 | RPL18 | 6141 | CCAGAAACCTGTATAACTGG | 22 | 3′ end | 1 | intron |
| in785887_a_1784 | RPL18 | 6141 | TGGCCAGAAACCTGTATAAC | 23 | 3′ end | 2 | exon |
| in785887_a_19 | RPL18 | 6141 | CGGAAAGAGAGAACGGGCTG | 24 | 5′ end | 46 | exon |
| in785887_a_21 | RPL18 | 6141 | TCCGGAAAGAGAGAACGGGC | 25 | 5′ end | 44 | exon |
| in785887_a_63 | RPL18 | 6141 | GCAAAGCGAGCTCACCATGA | 26 | 5′ end | 2 | exon |
| in785887_s_102 | RPL18 | 6141 | TAATCCGCTGCCATCCGCCG | 27 | 5′ end | 48 | intron |
| in785887_s_108 | RPL18 | 6141 | GCTGCCATCCGCCGTGGTTT | 28 | 5′ end | 54 | intron |
| in785887_s_109 | RPL18 | 6141 | CTGCCATCCGCCGTGGTTTT | 29 | 5′ end | 55 | intron |
| in785887_s_114 | RPL18 | 6141 | ATCCGCCGTGGTTTTGGGCT | 30 | 5′ end | 60 | intron |
| in785887_s_115 | RPL18 | 6141 | TCCGCCGTGGTTTTGGGCTA | 31 | 5′ end | 61 | intron |
| in785887_s_124 | RPL18 | 6141 | GTTTTGGGCTAGGGGCACGC | 32 | 5′ end | 70 | intron |
| in785887_s_127 | RPL18 | 6141 | TTGGGCTAGGGGCACGCTGG | 33 | 5′ end | 73 | intron |
| in785887_s_128 | RPL18 | 6141 | TGGGCTAGGGGCACGCTGGA | 34 | 5′ end | 74 | intron |
| in785887_s_1710 | RPL18 | 6141 | TCATGTGTTTGCCCCTTCAC | 35 | 3′ end | 61 | intron |
| in785887_s_1720 | RPL18 | 6141 | GCCCCTTCACTGGAAGGCTG | 36 | 3′ end | 51 | intron |
| in785887_s_1774 | RPL18 | 6141 | TCCCGTCCCTCCAGTTATAC | 37 | 3′ end | 3 | exon |
| in785887_s_65 | RPL18 | 6141 | ATCATGGTGAGCTCGCTTTG | 38 | 5′ end | 11 | intron |
| in785887_s_72 | RPL18 | 6141 | TGAGCTCGCTTTGCGGCGTT | 39 | 5′ end | 18 | intron |
| in785887_s_73 | RPL18 | 6141 | GAGCTCGCTTTGCGGCGTTC | 40 | 5′ end | 19 | intron |
| in785887_s_74 | RPL18 | 6141 | AGCTCGCTTTGCGGCGTTCG | 41 | 5′ end | 20 | intron |
| in785887_s_78 | RPL18 | 6141 | CGCTTTGCGGCGTTCGGGGC | 42 | 5′ end | 24 | intron |
| in785888_a_101 | RPL18 | 6141 | GACAAGACCCAGCGGCTCCC | 43 | 5′ end | 36 | intron |
| in785888_a_109 | RPL18 | 6141 | TCCAGACAGACAAGACCCAG | 44 | 5′ end | 44 | intron |
| in785888_a_483 | RPL18 | 6141 | CTTGAGGCATCCCCAGGCCA | 45 | 3′ end | 73 | intron |
| in785888_a_489 | RPL18 | 6141 | GCCCCGCTTGAGGCATCCCC | 46 | 3′ end | 67 | intron |
| in785888_a_499 | RPL18 | 6141 | TTTACATTCAGCCCCGCTTG | 47 | 3′ end | 57 | intron |
| in785888_a_524 | RPL18 | 6141 | ATGTACGTCGTAAGTTGTTC | 48 | 3′ end | 32 | intron |
| in785888_a_547 | RPL18 | 6141 | TTCCGGATCTTAGGGTGGGG | 49 | 3′ end | 9 | intron |
| in785888_a_550 | RPL18 | 6141 | ATCTTCCGGATCTTAGGGTG | 50 | 3′ end | 6 | intron |
| in785888_a_551 | RPL18 | 6141 | CATCTTCCGGATCTTAGGGT | 51 | 3′ end | 5 | intron |
| in785888_a_552 | RPL18 | 6141 | TCATCTTCCGGATCTTAGGG | 52 | 3′ end | 4 | intron |
| in785888_a_555 | RPL18 | 6141 | GCTTCATCTTCCGGATCTTA | 53 | 3′ end | 1 | intron |
| in785888_a_556 | RPL18 | 6141 | AGCTTCATCTTCCGGATCTT | 2 | 3′ end | 0 | intron |
| in785888_a_57 | RPL18 | 6141 | GCCACTCACCATCCGGGAAA | 54 | 5′ end | 8 | exon |
| in785888_a_58 | RPL18 | 6141 | AGCCACTCACCATCCGGGAA | 55 | 5′ end | 7 | exon |
| in785888_a_63 | RPL18 | 6141 | GGACCAGCCACTCACCATCC | 1 | 5′ end | 2 | exon |
| in785888_a_64 | RPL18 | 6141 | TGGACCAGCCACTCACCATC | 56 | 5′ end | 1 | exon |
| in785888_s_108 | RPL18 | 6141 | GCCGCTGGGTCTTGTCTGTC | 57 | 5′ nd | 54 | intron |
| in785888_s_113 | RPL18 | 6141 | TGGGTCTTGTCTGTCTGGAA | 58 | 5′ end | 59 | intron |
| in785888_s_116 | RPL18 | 6141 | GTCTTGTCTGTCTGGAAGGG | 59 | 5′ end | 62 | intron |
| in785888_s_487 | RPL18 | 6141 | GGCCTGGGGATGCCTCAAGC | 60 | 3′ end | 58 | intron |
| in785888_s_488 | RPL18 | 6141 | GCCTGGGGATGCCTCAAGCG | 61 | 3′ end | 57 | intron |
| in785888_s_545 | RPL18 | 6141 | ATCCTCCCCACCCTAAGATC | 62 | 3′ end | 0 | intron |
| in785888_s_56 | RPL18 | 6141 | TCCCTTTCCCGGATGGTGAG | 63 | 5′ end | 2 | intron |
| in785888_s_60 | RPL18 | 6141 | TTTCCCGGATGGTGAGTGGC | 64 | 5′ end | 6 | intron |
| in785888_s_74 | RPL18 | 6141 | AGTGGCTGGTCCAGAGAGCA | 65 | 5′ end | 20 | intron |
| in785888_s_83 | RPL18 | 6141 | TCCAGAGAGCACGGTAGACC | 66 | 5′ end | 29 | intron |
| in785888_s_94 | RPL18 | 6141 | CGGTAGACCTGGGAGCCGCT | 67 | 5′ end | 40 | intron |
| in785889_a_533 | RPL18 | 6141 | GTGGTCACCCAGGGGCTGCC | 68 | 3′ end | 55 | intron |
| in785889_a_541 | RPL18 | 6141 | ACCCCTGCGTGGTCACCCAG | 79 | 3′ end | 47 | intron |
| in785889_a_543 | RPL18 | 6141 | AGACCCCTGCGTGGTCACCC | 70 | 3′ end | 45 | intron |
| in785889_a_552 | RPL18 | 6141 | TGGCGGGTCAGACCCCTGCG | 71 | 3′ end | 36 | intron |
| in785889_a_569 | RPL18 | 6141 | GGTGGAGAGGACAAGGCTGG | 72 | 3′ end | 19 | intron |
| in785889_a_572 | RPL18 | 6141 | CCTGGTGGAGAGGACAAGGC | 73 | 3′ end | 16 | intron |
| in785889_a_576 | RPL18 | 6141 | CATACCTGGTGGAGAGGACA | 74 | 3′ end | 12 | intron |
| in785889_a_582 | RPL18 | 6141 | AGTGCACATACCTGGTGGAG | 75 | 3′ end | 6 | intron |
| in785889_a_587 | RPL18 | 6141 | CGCGCAGTGCACATACCTGG | 76 | 3′ end | 1 | intron |
| in785889_a_59 | RPL18 | 6141 | CGCCAGCTCACCTTCAGTTT | 77 | 5′ end | 6 | exon |
| in785889_a_590 | RPL18 | 6141 | TCACGCGCAGTGCACATACC | 78 | 3′ end | 2 | exon |
| in785889_a_60 | RPL18 | 6141 | CCGCCAGCTCACCTTCAGTT | 79 | 5′ end | 5 | exon |
| in785889_a_96 | RPL18 | 6141 | ACAGTACAGCAAGGGTCTGA | 80 | 5′ end | 31 | intron |
| in785889_s_504 | RPL18 | 6141 | CTGCTGCGCCAAGGCAGTGG | 81 | 3′ end | 73 | intron |
| in785889_s_505 | RPL18 | 6141 | TGCTGCGCCAAGGCAGTGGA | 82 | 3′ end | 72 | intron |
| in785889_s_515 | RPL18 | 6141 | AGGCAGTGGAGGGTGAGTCC | 83 | 3′ end | 62 | intron |
| in785889_s_526 | RPL18 | 6141 | GGTGAGTCCTGGCAGCCCCT | 84 | 3′ end | 51 | intron |
| in785889_s_539 | RPL18 | 6141 | AGCCCCTGGGTGACCACGCA | 85 | 3′ end | 38 | intron |
| in785889_s_540 | RPL18 | 6141 | GCCCCTGGGTGACCACGCAG | 86 | 3′ end | 37 | intron |
| in785889_s_61 | RPL18 | 6141 | CAAACTGAAGGTGAGCTGGC | 87 | 5′ end | 7 | intron |
| in785889_s_62 | RPL18 | 6141 | AAACTGAAGGTGAGCTGGCG | 88 | 5′ end | 8 | intron |
| in785889_s_63 | RPL18 | 6141 | AACTGAAGGTGAGCTGGCGG | 89 | 5′ end | 9 | intron |
| in785889_s_68 | RPL18 | 6141 | AAGGTGAGCTGGCGGGGGCT | 90 | 5′ end | 14 | intron |
| in785890_a_130 | RPL18 | 6141 | TCTGGCCTCCCAGATCCAGG | 91 | 3′ end | 67 | intron |
| in785890_a_148 | RPL18 | 6141 | GGGATCTGGCGCCCAGCTTC | 92 | 3′ end | 49 | intron |
| in785890_a_162 | RPL18 | 6141 | AACCGGGTGAGACAGGGATC | 93 | 3′ end | 35 | intron |
| in785890_a_168 | RPL18 | 6141 | AAGGAGAACCGGGTGAGACA | 94 | 3′ end | 29 | intron |
| in785890_a_169 | RPL18 | 6141 | GAAGGAGAACCGGGTGAGAC | 95 | 3′ end | 28 | intron |
| in785890_a_191 | RPL18 | 6141 | CTTGCGAGGACCTAGGGAAG | 96 | 3′ end | 6 | intron |
| in785890_a_192 | RPL18 | 6141 | CCTTGCGAGGACCTAGGGAA | 97 | 3′ end | 5 | intron |
| in785890_a_193 | RPL18 | 6141 | CCCTTGCGAGGACCTAGGGA | 98 | 3′ end | 4 | intron |
| in785890_a_197 | RPL18 | 6141 | TCGGCCCTTGCGAGGACCTA | 99 | 3′ end | 0 | intron |
| in785890_a_198 | RPL18 | 6141 | CTCGGCCCTTGCGAGGACCT | 100 | 3′ end | 1 | exon |
| in785890_a_29 | RPL18 | 6141 | ACAGCCCTTAGGGGAGTCCA | 101 | 5′ end | 36 | exon |
| in785890_a_30 | RPL18 | 6141 | CACAGCCCTTAGGGGAGTCC | 102 | 5′ end | 35 | exon |
| in785890_a_60 | RPL18 | 6141 | CGTATCACTCACCGGAGAGC | 103 | 5′ end | 5 | exon |
| in785890_a_68 | RPL18 | 6141 | GTCGACCACGTATCACTCAC | 104 | 5′ end | 3 | intron |
| in785890_s_115 | RPL18 | 6141 | ACTGGCAGCCTTCACCCTCC | 105 | 3′ end | 71 | intron |
| in785890_s_121 | RPL18 | 6141 | AGCCTTCACCCTCCTGGATC | 106 | 3′ end | 65 | intron |
| in785890_s_122 | RPL18 | 6141 | GCCTTCACCCTCCTGGATCT | 107 | 3′ end | 64 | intron |
| in785890_s_136 | RPL18 | 6141 | GGATCTGGGAGGCCAGAAGC | 108 | 3′ end | 50 | intron |
| in785890_s_137 | RPL18 | 6141 | GATCTGGGAGGCCAGAAGCT | 109 | 3′ end | 49 | intron |
| in785890_s_160 | RPL18 | 6141 | CGCCAGATCCCTGTCTCACC | 110 | 3′ end | 26 | intron |
| in785890_s_63 | RPL18 | 6141 | GCTCTCCGGTGAGTGATACG | 111 | 5′ end | 9 | intron |
| in785890_s_70 | RPL18 | 6141 | GGTGAGTGATACGTGGTCGA | 112 | 5′ end | 16 | intron |
| in785890_s_71 | RPL18 | 6141 | GTGAGTGATACGTGGTCGAC | 113 | 5′ end | 17 | intron |
| in785890_s_76 | RPL18 | 6141 | TGATACGTGGTCGACGGGTT | 114 | 5′ end | 22 | intron |
| in785890_s_97 | RPL18 | 6141 | GGACTGAGCTGTGTGGCTAC | 115 | 5′ end | 43 | intron |
| in785891_a_435 | RPL18 | 6141 | AGGCCATTGTGGAGTGGCAC | 116 | 3′ end | 59 | intron |
| in785891_a_492 | RPL18 | 6141 | GAGCGGACGTAGGGTCTGTG | 117 | 3′ end | 2 | intron |
| in785891_a_493 | RPL18 | 6141 | GGAGCGGACGTAGGGTCTGT | 118 | 3′ end | 1 | intron |
| in785891_a_494 | RPL18 | 6141 | TGGAGCGGACGTAGGGTCTG | 119 | 3′ end | 0 | intron |
| in785891_a_53 | RPL18 | 6141 | CTCACTTGGTGTGGCTGTGC | 120 | 5′ end | 12 | exon |
| in785891_a_54 | RPL18 | 6141 | ACTCACTTGGTGTGGCTGTG | 121 | 5′ end | 11 | exon |
| in785891_a_67 | RPL18 | 6141 | CTGGGGGCCTGATACTCACT | 122 | 5′ end | 2 | intron |
| in785891_s_432 | RPL18 | 6141 | GTTCCTGTGCCACTCCACAA | 123 | 3′ end | 51 | intron |
| in785891_s_60 | RPL18 | 6141 | AGCCACACCAAGTGAGTATC | 124 | 5′ end | 6 | intron |
| in785892_a_1317 | RPL18 | 6141 | CACTCCCTGTGGGGGTGAAG | 125 | 3′ end | 22 | intron |
| in785892_a_1325 | RPL18 | 6141 | CGGATGTCCACTCCCTGTGG | 126 | 3′ end | 3 | intron |
| in785892_a_1326 | RPL18 | 6141 | GCGGATGTCCACTCCCTGTG | 127 | 3′ end | 2 | intron |
| in785892_a_1327 | RPL18 | 6141 | GGCGGATGTCCACTCCCTGT | 128 | 3′ end | 1 | intron |
| in785892_a_1328 | RPL18 | 6141 | TGGCGGATGTCCACTCCCTG | 129 | 3′ end | 0 | intron |
| in785892_s_1263 | RPL18 | 6141 | TTTCAGAAATAAGTAATAAT | 130 | 3′ end | 54 | intron |
| in785892_s_1274 | RPL18 | 6141 | AGTAATAATTGGCTATGGTT | 131 | 3′ end | 43 | intron |
| in785892_s_1276 | RPL18 | 6141 | TAATAATTGGCTATGGTTGG | 132 | 3′ end | 41 | intron |
| in785892_s_1283 | RPL18 | 6141 | TGGCTATGGTTGGGGGTAAT | 133 | 3′ end | 34 | intron |
| in785892_s_1284 | RPL18 | 6141 | GGCTATGGTTGGGGGTAATT | 134 | 3′ end | 33 | intron |
| in785892_s_1291 | RPL18 | 6141 | GTTGGGGGTAATTGGGTCCA | 135 | 3′ end | 26 | intron |
| in785892_s_1312 | RPL18 | 6141 | GGTTGCCTCTTCACCCCCAC | 136 | 3′ end | 5 | intron |
| in785892_s_1313 | RPL18 | 6141 | GTTGCCTCTTCACCCCCACA | 137 | 3′ end | 4 | intron |
| in785892_s_1318 | RPL18 | 6141 | CTCTTCACCCCCACAGGGAG | 138 | 3′ end | 1 | exon |
| in785892_s_1336 | RPL18 | 6141 | AGTGGACATCCGCCATAACA | 139 | 3′ end | 19 | exon |
| in785893_a_106 | RPL18 | 6141 | GGATCTGCAAGTCAGACCTG | 140 | 5′ end | 41 | intron |
| in785893_a_108 | RPL18 | 6141 | GAGGATCTGCAAGTCAGACC | 141 | 5′ end | 43 | intron |
| in785893_a_130 | RPL18 | 6141 | GCTTGGTGCCAGCACTAGAA | 142 | 5′ end | 65 | intron |
| in785893_a_82 | RPL18 | 6141 | GACCCTTCCCAAAGACCTCA | 143 | 5′ end | 17 | intron |
| in785893_a_83 | RPL18 | 6141 | TGACCCTTCCCAAAGACCTC | 144 | 5′ end | 18 | intron |
| in785893_s_58 | RPL18 | 6141 | GCTGTTGGTCAAGGTGAGGC | 145 | 5′ end | 4 | intron |
| in785893_s_59 | RPL18 | 6141 | CTGTTGGTCAAGGTGAGGCT | 146 | 5′ end | 5 | intron |
| in785893_s_74 | RPL18 | 6141 | AGGCTGGGCCCTGAGGTCTT | 147 | 5′ end | 20 | intron |
| in785893_s_75 | RPL18 | 6141 | GGCTGGGCCCTGAGGTCTTT | 148 | 5′ end | 21 | intron |
| in785893_s_79 | RPL18 | 6141 | GGGCCCTGAGGTCTTTGGGA | 149 | 5′ end | 25 | intron |
| in785893_s_90 | RPL18 | 6141 | TCTTTGGGAAGGGTCACCCC | 150 | 5′ end | 36 | intron |

The cell library harbouring these sgRNAs were constructed through lentiviral delivery at an MOI of <0.3 in Cas9-expressing HeLa and Huh7.5 cells²⁸, with a minimum coverage of 400×. 72 hours after viral infection, the cells were sorted by FACS (BD) for mCherry⁺. The control cells (2.4×10⁶) of each library were collected for genomic DNA extraction using the DNeasy Blood and Tissue kit (QIAGEN 69506), and the experimental cells were continuously cultured for 15 days before genomic DNA extraction. For each replicate, the lentivirally integrated sgRNA-coding regions were PCR-amplified by TransTaq HiFi DNA Polymerase (TransGen AP131-13), and further purified with DNA Clean & Concentrator-25 (Zymo Research Corporation D4034) as previously described^{4,9}. The resulting libraries were prepared for high-throughput sequencing analysis (Illumina HiSeq2500) using NEBNext Ultra DNA Library Prep Kit for Illumina (NEB E7370L).

4. Design and Construction of the Genome-Scale Human lncRNA Library

LncRNA annotations were retrieved from GENCODE dataset V20 which contains 14,470 lncRNAs. In this dataset, 2,477 lncRNAs without splice sites were removed in the first filtering process. For the rest lncRNAs, all potential 20-nt sgRNAs targeting −10-bp to +10-bp regions surrounding every 5′ SD site and 3′ SA site were designed. To ensure cleavage efficiency and specificity, we only kept sgRNAs with at least 2 mismatches to other loci in genome, whose GC content is between 20% and 80%, and removed those sgRNAs that contain ≥4-bp homopolymeric stretch of T nucleotides. To achieve the best coverage, certain sgRNAs with 1-bp or 0-bp mismatches to other loci were retained as long as they do not target any essential genes of K562 cell line¹⁵ and the total number of mismatched sites is less than 2. Total of 126,773 sgRNAs targeting 10,996 lncRNAs were ultimately synthesized. In the library, we also included 500 non-targeting sgRNAs in human genome as negative controls, and 350 sgRNAs targeting 36 essential ribosomal genes as positive controls. The oligonucleotides were synthesized using the CustmoArray 90K array chips (CustmoArray, Inc.), and the library construction was the same as described above.

5. Genome-Scale lncRNA Screening

A total of 5×10⁸ K562 cells were plated onto the 175 cm² flasks (Corning 431080) for each of two replicates. Cells were infected with sgRNA library lentiviruses at an MOI of less than 0.3 (1000× coverage) in 24 hrs. 48 hrs post infection, the library cells were subjected to puromycin treatment (3 μg/ml; Solarbio P8230) for two days. For each replicate, a total of 1.3×10⁸ cells were collected as the Day-0 control samples for genome extraction. 30 days post viral infection, 1.3×10⁸ experimental cells were isolated for genome extraction and NGS analysiso.

### 6. Computational Analysis of Screens

Sequencing reads were mapped to hg38 reference genome and decoded by home-made scripts. sgRNA counts from two replicates were quantile normalized, then average counts and fold changes between experimental and control groups were calculated. 1000 negative control genes were generated by randomly sampling 10 negative control sgRNAs with replacement per gene. Noisy sgRNAs were then filtered based on the following criteria: if a sgRNA's fold change was lower than mean fold change of positive control sgRNAs in one replicate and higher than mean fold change of negative control sgRNAs in another replicate, the sgRNA was regarded as a noisy sgRNA for filtering. For each lncRNA after noise filtering, we compared the fold change of sgRNAs with negative control by Wilcox test, and corrected the P values using empirical distribution generated by negative control genes to reduce false positive rate. We ultimately defined screen score as: screen score=scale(−log₁₀(adjusted p-value))+|scale(log₂(sgRNA fold change))|. We designated those hits with screen score higher than 2 as essential lncRNAs.

7. Validation of lncRNA Hits

The two top-ranking sgRNAs for validation by splicing strategy were selected from library, which had at least 2 mismatches to any other loci in the genome. For the pgRNA deletion strategy, pgRNAs were designed to delete the promoter and the first exon of each lncRNA. We designed gRNA pairs according to the following criteria: (1) one sgRNA targets the 2.5-3.5 kb regions upstream the transcription start site (TSS) and the other one targets the 0.2-1.5 kb regions downstream the TSS: (2) avoid overlapping with any exons or promoters of coding or nocoding genes. For each sgRNA of the pairs, we further ensured that (1) the GC content is between 45% and 70%, (2) the sgRNA does not include ≥4-bp homopolymer stretch, and (3) the sgRNA contains more than 2 mismatches to any other loci in human genome. We included some sgRNAs with 2 mismatches to other loci, but the number of off-target sites is less than 2.

All the sgRNAs or pgRNAs targeting the selected lncRNAs to be validated were individually cloned into the lentiviral vector with a CMV promoter-driven EGFP marker. After virus packaging, the sgRNA or pgRNA lentivirus was transduced into K562 or GM12878 cells at an MOI of <1.0. The cell proliferation assay was previously described⁹.

### 8. RNA Sequencing and Data Analysis

Two sgRNAs targeting the splice sites of lncRNA MIR17HG and BMS1P20 were individually cloned into the lentiviral vector with an EGFP marker. The sgRNAs were delivered into K562 or GM12878 cells by lentiviral infection at an MOI of <1. 2×10⁶ EGFP positive cells of K562 or GM12878 were sorted by FACS 5 days post infection. Total RNA of each sample was extracted using RNeasy Mini Kit (QIAGEN 79254), and the RNA-seq libraries were prepared following the NEBNext PolyA mRNA Magnetic Isolation Module (NEB E7490S), NEBNext RNA First Strand Synthesis Module (NEB E7525S), NEBNext mRNA Second Strand Synthesis Module (NEB E6111S) and NEBNext Ultra DNA Library Prep Kit for Illumina (NEB E7370L). All samples were subjected to NGS analysis using the Illumina HiSeq X Ten platform (Genetron Health). Deep sequencing reads were mapped to hg38 reference genome and gene expression was quantified by RSEM v1.2.25³⁰. Differential expression analysis was conducted by EBSeq version 1.10.0³¹ and differentially expressed genes were selected from those that had adjusted P value <0.05 and absolute log₂(fold change) >3. Gene Ontology and KEGG analysis was conducted by DAVID 6.8³².

### Result

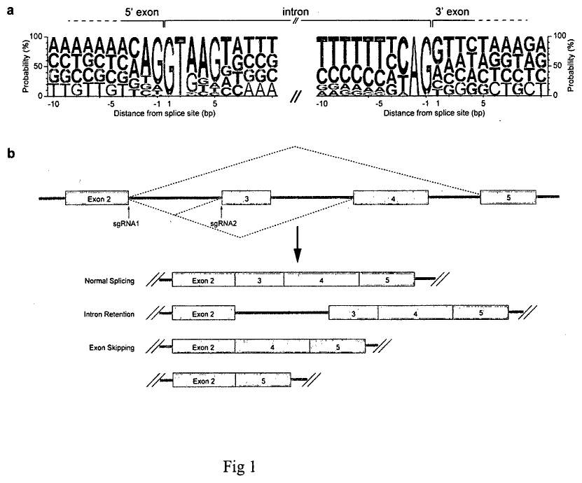

In consistence with the common knowledge that there are conserved sequences marking the splice sites, our bioinformatics analysis using Weblogo3 tools³³ showed that about 99% intronic regions in human genome are flanked by GT at the 5′ splice donor (SD) sites and AG at the 3′ splice acceptor (SA) sites. It is worthy of note that AG sequences are predominantly present as the last two bases of exons just upstream of the SD sites (FIG. 1a). To verify the effectiveness of a sgRNA in producing exon skipping and/or intron retention, we designed sgRNAs targeting either SD or SA sites of two ribosomal genes, RPL18 and RPL11, both of which are indispensable for cell growth and proliferation. In HeLa cells stably expressing Cas9 and OCT1 genes⁴, sgRNA1_{RPL18} targeting an SD site and sgRNA2_{RPL18} targeting an SA site successfully generated intron 3 retention and exon 4 skipping on RPL18 loci in genome, respectively, which were confirmed by both reverse transcription-PCR (RT-PCR) and Sanger sequencing analysis. The same results were obtained from a similar attempt on RPL11 genes, in which sgRNA3_{RPL11} and sgRNA4_{RPL11} produced intron 2 retention and exon 4 skipping on RPL11 loci, respectively. FIG. 1b shows the intron retention or exon skipping induced by sgRNAs targeting splicing donor (SD) or splicing acceptor (SA) site.

To further assess the power of splicing-targeting in CRISPR screen, we designed a saturation library targeting splice sites of 79 ribosomal genes, most of which were essential for cellular growth in various cell lines²⁹. This library contained 5,788 sgRNAs whose cutting sites are within −50-bp to +75-bp surrounding every 5′ SD site and −75-bp to +50-bp surrounding every 3′ SA site of these 79 genes (see Table 1 for the examples of sgRNA).

The cell libraries harbouring these sgRNAs were constructed through lentiviral delivery at an MOI (multiplicity of infection) of <0.3 in Cas9-expressing HeLa and Huh7.5 cells14. The screening was performed through prolonged cell culturing of library cells spanning 15 days, and the sgRNAs leading to cell viability drops were deciphered based on NGS analysis.

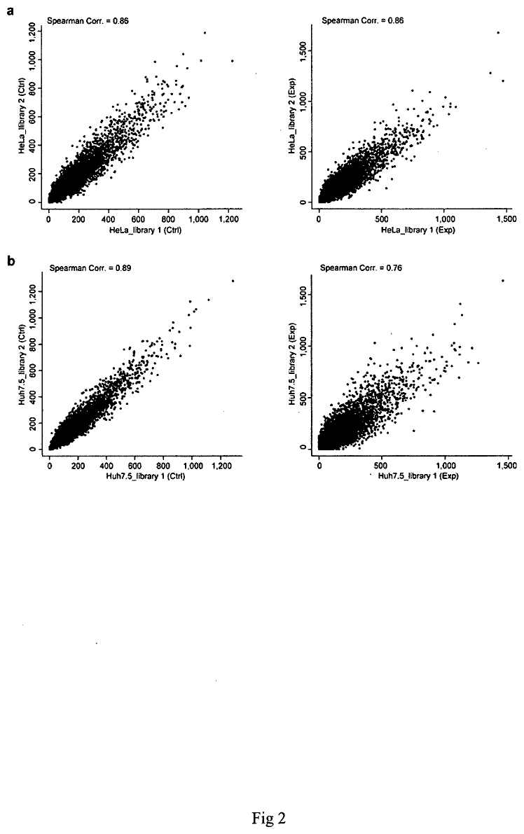

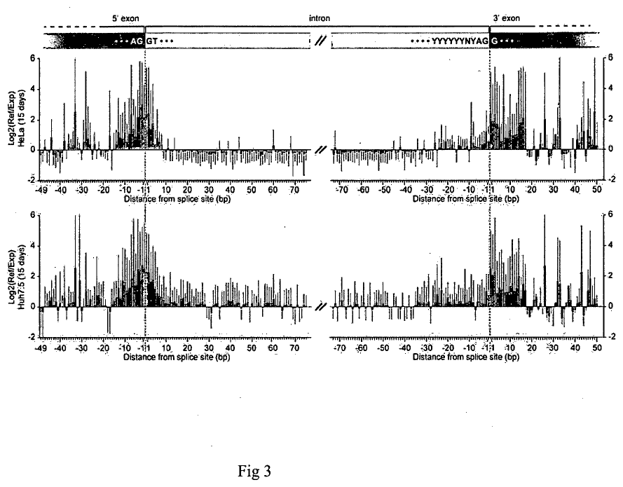

By calculating the log₂ fold change of sgRNAs between 15-day experimental (Exp) and control (Ctrl) samples, we ranked all sgRNAs and aligned them according to their distances in base pair (bp) between sgRNA-cutting sites and their corresponding SD or SA sites. The Spearman correlation between the biological replicates of Ctrl and Exp in both HeLa and Huh7.5 cells showed that all results were highly reproducible (FIG. 2). To manifest the effectiveness of splicing targeting on gene disruption, we merged all SD site-targeting data and SA site-targeting data, and arranged them according to their physical distances relevant to SD or SA sites (FIG. 3 and FIG. 1d). It became evident that sgRNAs affecting splice sites outperformed those targeting only exonic regions in both HeLa and Huh7.5 cells. The closer the distances from sgRNAs' cutting sites to splice sites, the better their effects on gene disruption, with peak points slightly towards the exons for both SD and SA cases (FIG. 3 FIG. 1d). In comparison, vast majority of sgRNAs targeting introns were rarely depleted throughout the screens, suggesting that they had little effects on gene disruption and consequently the loss of gene functions on cell viability. The only exceptions were those sgRNAs targeting intronic regions close to SA sites, which include branchpoints followed by polypyrimidine tracts that have been known for their involvement in RNA splicing^{34,35}.

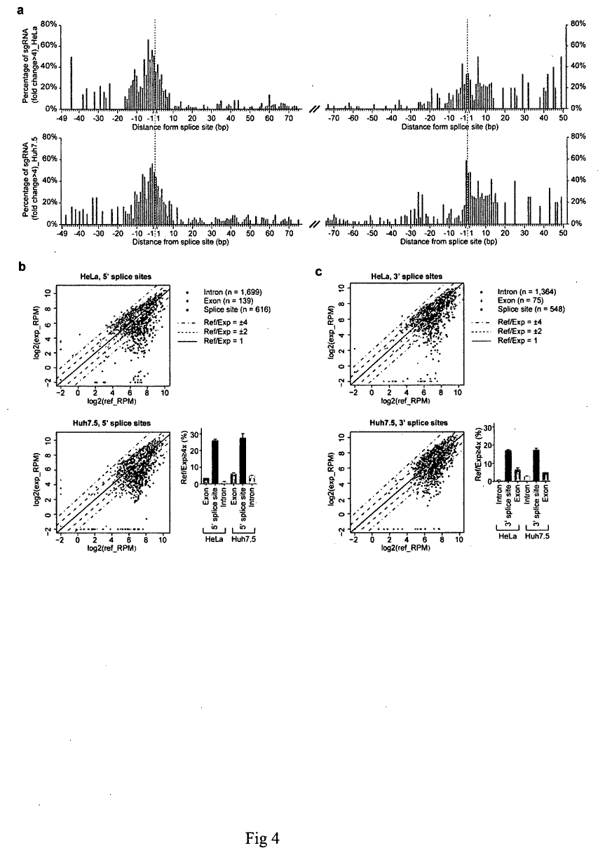

As the numbers of sgRNAs designed for any locus were not equal, we compared the percentages of high-efficient (over 4-fold dropout) sgRNAs at every locus for fair comparison. With such normalization, we further confirmed that both SD- and SA-targeting sgRNAs were vastly superior to those targeting only exonic regions (FIG. 4a). To better quantify our results, we classified all sgRNAs into three categories: intron-targeting (cutting sites of sgRNAs are within introns and at least 30-bp away from SD or SA sites), exon-targeting (cutting sites of sgRNAs are within exons and at least 30-bp away from SD or SA sites), and splicing-targeting (cutting sites of sgRNAs are between −10-bp to +10-bp flanking SD or SA sites; − and + refer to intronic and exonic direction, respectively). In both HeLa and Huh7.5 cells, the percentages of sgRNAs leading to over 2- or 4-fold dropouts were much higher in splicing-targeting than the other two categories (FIG. 4b, 4c).

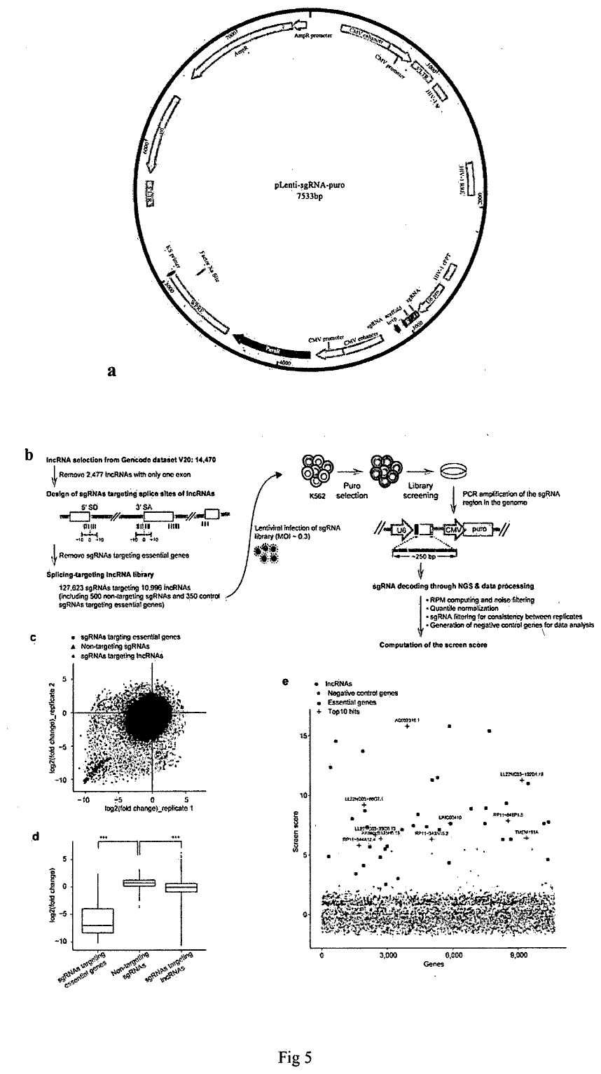

Based on above results, we inferred that this strategy should be universally applicable for coding genes and noncoding RNAs because RNA splicing is a well conserved mechanism for both. Assuming that targeting splice sites would potentially enable functional disruptions of lncRNAs in human cells through either exon skipping and/or intron retention, we designed and constructed a special splicing-targeting sgRNA library to establish the genome-scale and functional screening of lncRNAs. Among 14,470 lncRNAs retrieved from GENCODE dataset V20, we first filtered out 2,477 lacking splice sites. We abided by several other rules: all sgRNAs' cutting sites are within −10-bp to +10-bp surrounding splice sites, and sgRNAs are predicted to have high cleavage activity^{29,36,37} without off-targeting to any known essential gene¹⁵ (see Methods). We ultimately generated a library containing 126,773 sgRNAs targeting 10,996 unique lncRNAs. Together with 500 non-targeting control sgRNAs and 350 sgRNAs targeting essential ribosomal genes, we constructed the cell library in K562 cells engineered to stably express Cas9 protein (FIG. 5a and FIG. 2a). The cell library was made through lentiviral transduction at a low MOI of <0.3. We continued to culture the library cells for 30 days post infection to screen for those lncRNAs affecting cell growth and proliferation. NGS analysis was subsequently employed for sgRNA deciphering^{4,9} (FIG. 5b).

After 30-day culturing, sgRNAs targeting lncRNAs and essential genes were both depleted compared with the non-targeting sgRNAs (FIG. 5c, 5d FIG. 2b, c), indicating their effects on cell viability or proliferation. For each lncRNA, we computed the fold changes of sgRNAs and obtained their P values by comparing with non-targeting sgRNAs through Wilcoxon test. We randomly sampled non-targeting sgRNAs to generate “negative control genes”, thus correcting the lncRNA genes' P values by their distribution. For each lncRNA, a screen score was computed through combining the mean fold change and corrected P values (see Methods). Total of 243 lncRNA candidates were thus selected based on a threshold of the screen score of 2, whose depletion would lead to cell growth inhibition or cell death in K562 line (FIG. 5e FIG. 2d). According to the screen score, all 36 essential genes were significantly enriched in the ranking list of negatively selected genes, indicating the reliability of the screening approach and the data analysis method.

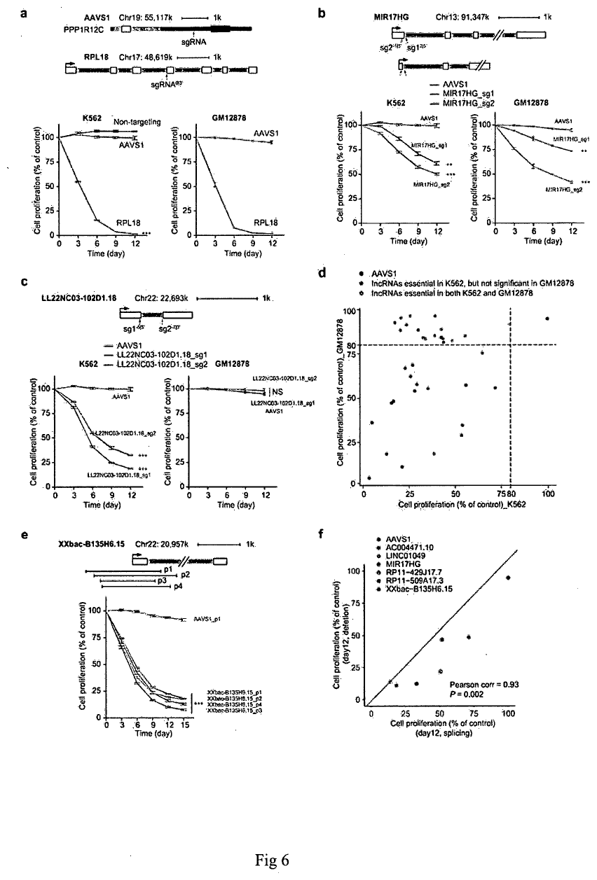

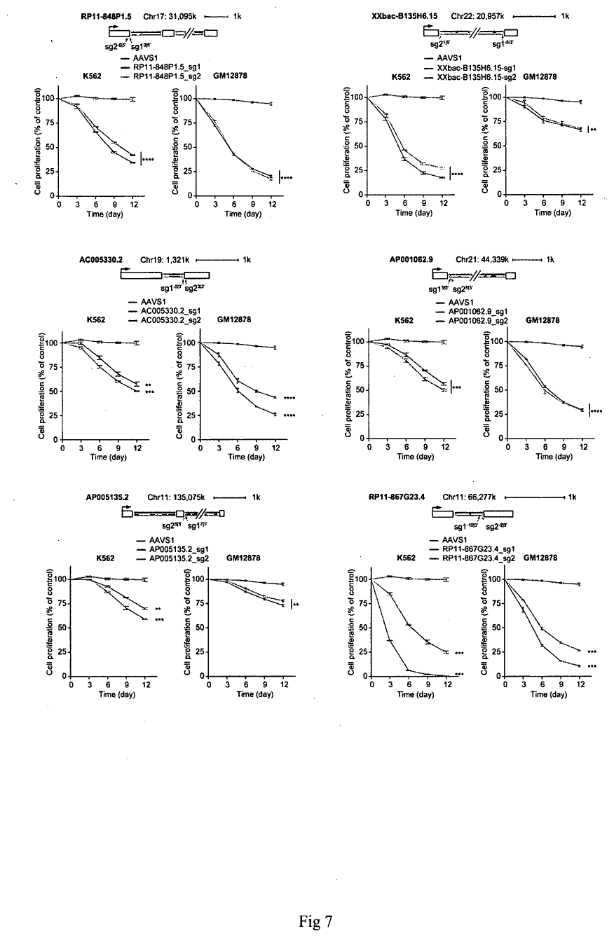

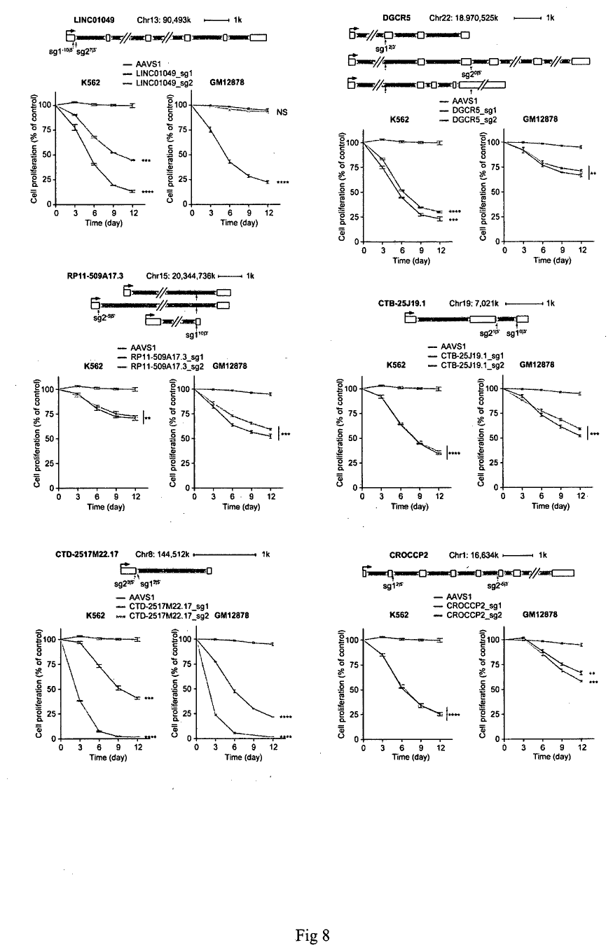

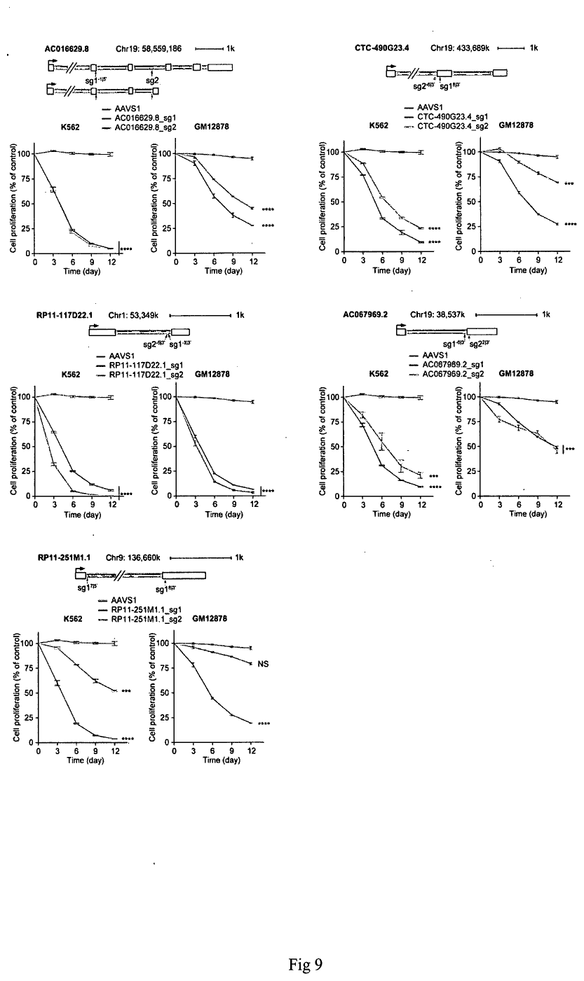

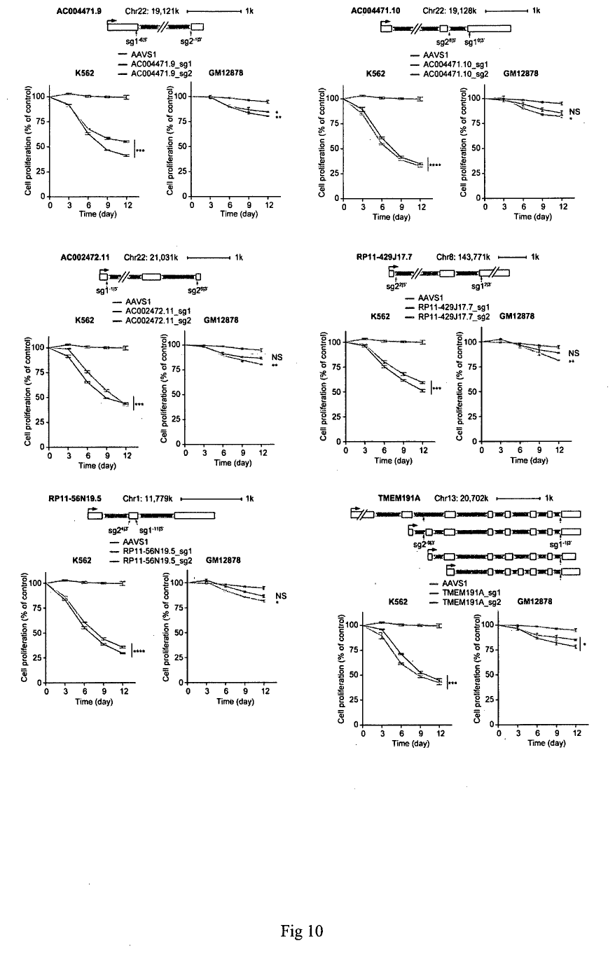

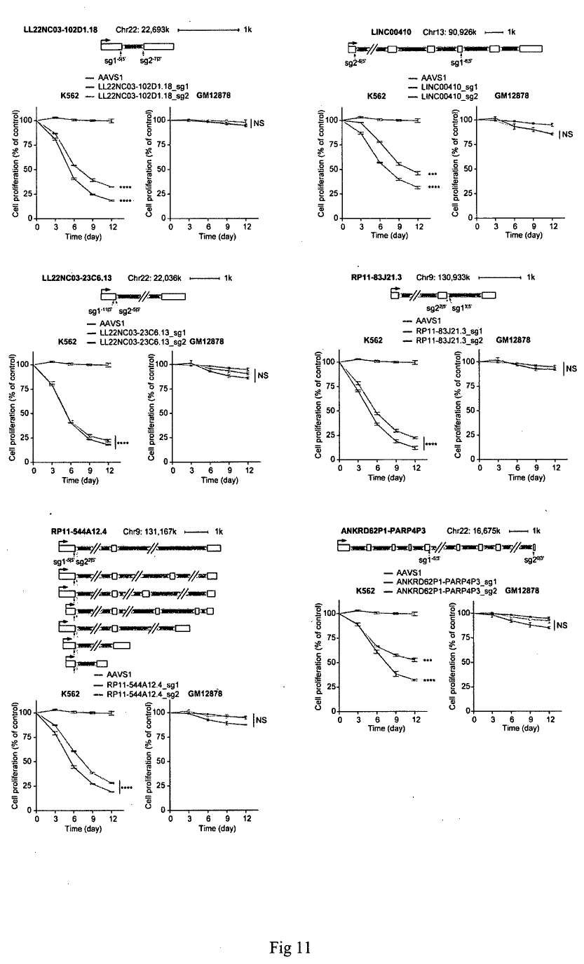

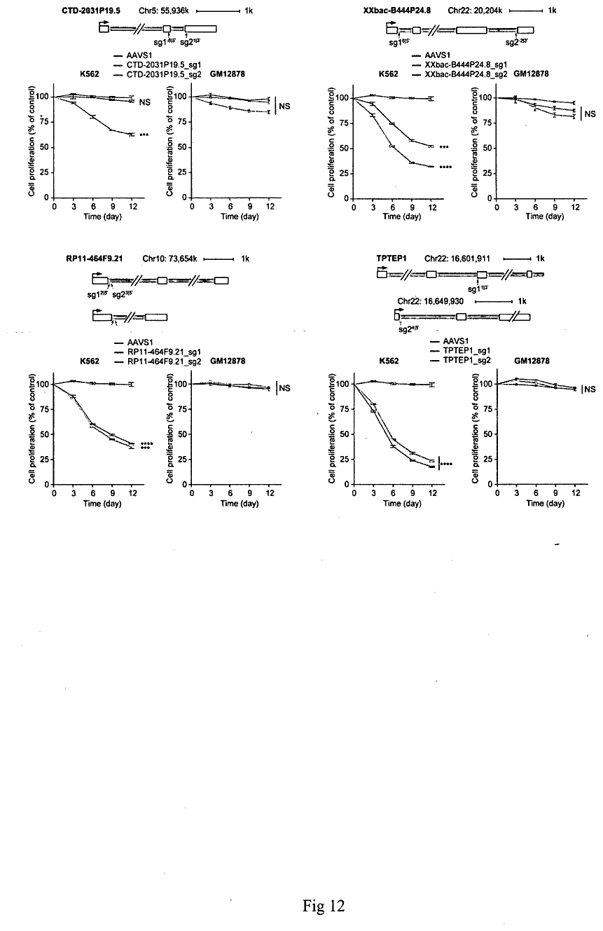

From the negatively selected lncRNAs whose corresponding sgRNAs were consistently depleted in two replicates, we chose 35 top-ranking lncRNA genes for further validation. For each candidate, we cloned the two top-ranking sgRNAs obtained from library screen into a lentiviral backbone with an EGFP selection marker. A non-targeting sgRNA and a sgRNA targeting the non-functional adeno-associated virus integration site 1 (AAVS1) locus were chosen as negative controls, and an sgRNA targeting the ribosomal gene RPL18 was also included as the positive control (FIG. 6a FIG. 3a). Each sgRNA was transduced into K562 cells, and the cell proliferation was quantified based on the percentage changes of EGFP-positive cells. To further explore the difference of lncRNA functions between cancer and normal cells, we included lymphoblastoid cell GM12878 for validation, which has a relatively normal karyotype and belongs to the Tier 1 ENCODE cell line as K562^{24,25}. Remarkably, all sgRNAs targeting the 35 top-ranking lncRNA loci effectively led to the inhibition of cell proliferation in K562 cells (FIG. 6b, c FIG. 3b, c, and FIG. 7-12). Among them, 18 lncRNAs appeared essential for the growth of GM12878 cells as well (FIG. 6b and FIG. 7-10 FIG. 3b), while 6 and 11 lncRNA hits showed weak (FIG. 10) and no detectable effects (FIG. 6c and FIG. 11-12 FIG. 3c) on cell viability in GM12878, respectively. These results suggest that there exists cell type specificity. In sum, about half of lncRNAs essential in K562 had no significant effects on the growth of GM12878 cells, representing unique biomarkers for cancerous cells with therapeutic potential (FIG. 6d FIG. 3d).

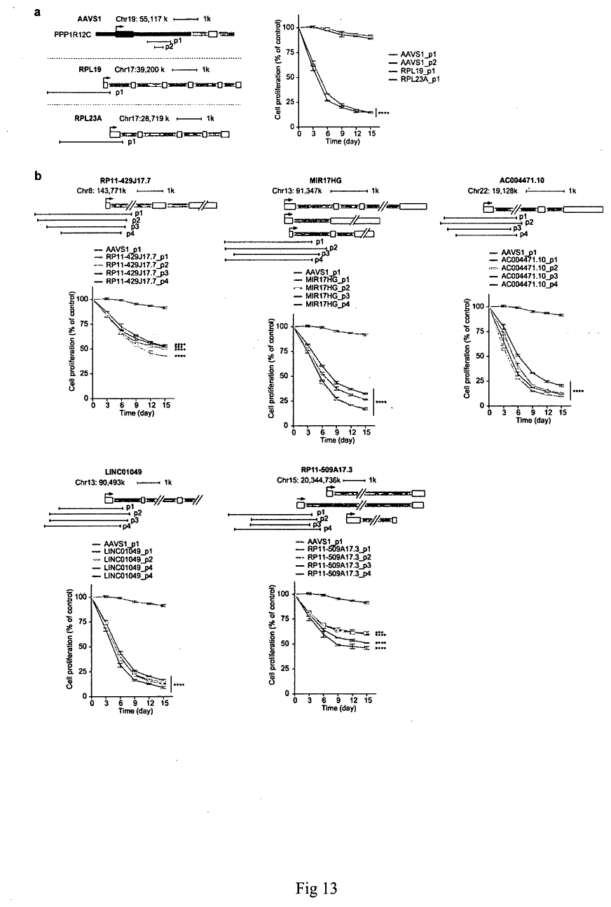

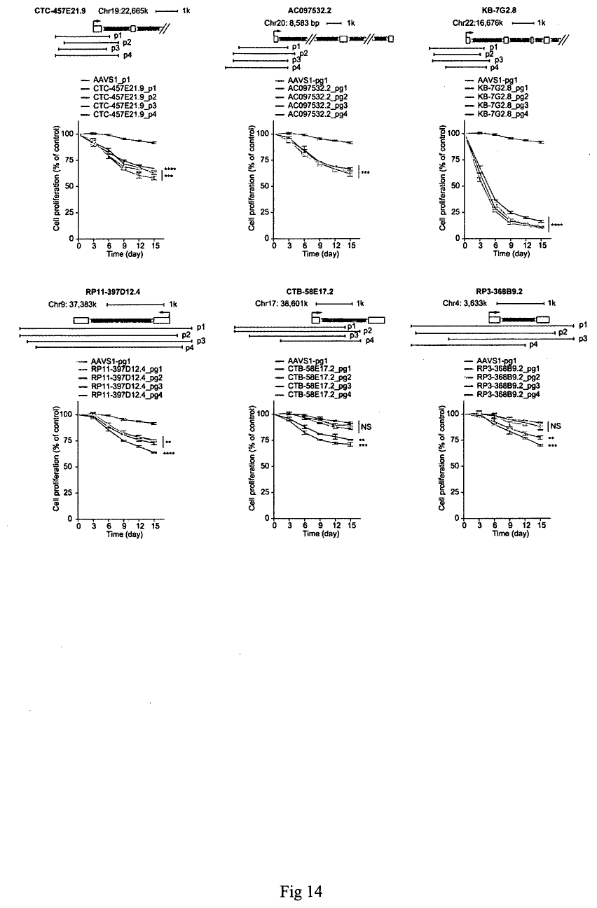

To further verify our validation assay as well as the screening strategy, both of which relied on splicing-perturbation, we chose the pgRNA-mediated deletion method9 to independently investigate the roles of lncRNA hits from our screen. We selected 6 lncRNAs from the validated 35 hits, and another 6 candidates from the top hits which were not included in above validation because their top-ranking splicing-targeting sgRNAs had certain off-target possibility. Four pgRNAs were designed for each of these 12 lncRNAs, deleting their promoters and first exons (see Methods). AAVS1 locus or ribosomal genes RPL19 and RPL23A were chosen for pgRNA targeting as negative control or positive controls, respectively (FIG. 13a). Through the cell proliferation assay, 6 lncRNAs from the 35 validated hits showed reproducible phenotypes as validated by the splicing-targeting strategy (FIG. 6e and FIG. 13b FIG. 3e). Validation results from splicing-targeting correlated well with those from deletion strategy (correlation coefficient=0.93, P=0.002) (FIG. 6f FIG. 3f), indicating that splicing-targeting is a reliable and robust approach for lncRNA gene disruption. Similarly, we demonstrated that the other 6 lncRNA candidates were also important for the growth of K562 cells (FIG. 14). Thus far, all 41 lncRNA hits were confirmed to be critically important for K562 cell growth and proliferation.

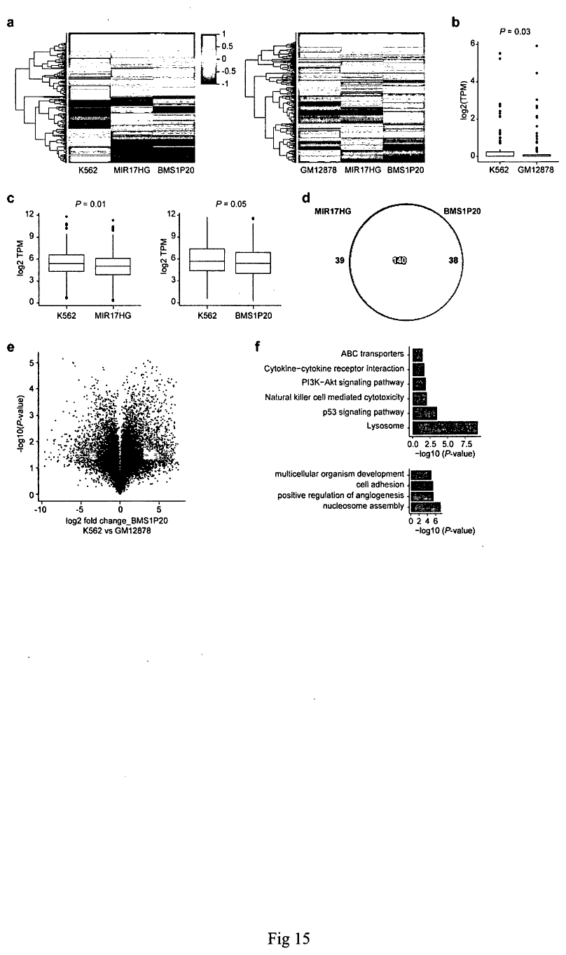

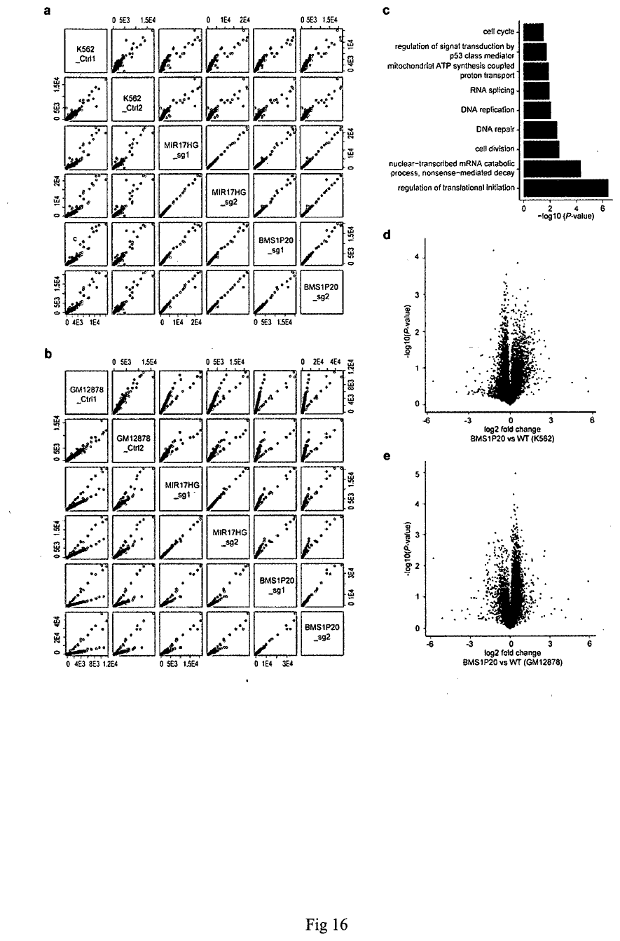

To better understand the mechanisms leading to these varied phenotypes in K562 and GM12878 cells, we further explored the functions of lncRNA MIR17HG which was essential for both cell lines (FIG. 6b FIG. 3b), and BMS1P20 which was essential for cell viability only in K562 but not in GM12878 (FIG. 6c FIG. 3c). We performed RNA-seq analysis of both K562 and GM12878 cells, with and without MIR17HG or BMS1P20 knockouts. We disrupted each lncRNA with two sgRNAs targeting their splice sites, whose effectiveness was confirmed in validation assays (FIG. 6b, c FIG. 3b, c). The expression levels of the top 500 genes showing variance between control and sgRNA-targeting samples were evaluated and different expression patterns were observed after knocking out the two lncRNAs (FIG. 15a FIG. 4a). For both lncRNAs in each cell line, the two sgRNAs targeting the same splice site with similar changes in expression patterns were shown (FIG. 16a, b). The overall expression levels of the top 100 essential lncRNAs identified from K562 cells were higher in the wild-type K562 cells than in GM12878 cells (P=0.03, FIG. 15b FIG. 4b).

In K562 cell line, changing the splicing pattern of MIR17HG down-regulated 179 known essential genes¹⁵ which affect cell growth and proliferation (P=0.01, FIG. 15c FIG. 4c), and disruption of BMS1P20 down-regulated 178 known essential genes¹⁵ (P=0.05, FIG. 15c FIG. 4c), suggesting the possible mechanisms how these two lncRNAs affect the growth of K562 cells. Surprisingly, MIR17HG and BMS1P20 affect 140 common essential genes in K562 cells (FIG. 15d FIG. 4d), albeit that they play distinct roles in GM12878 cells. These conserved genes were enriched in several essential pathways such as regulation of translational initiation, cell division and DNA repair (FIG. 16c). For BMS1P20, disruption of this lncRNA up- or down-regulated the expression of a series of coding genes in both K562 and GM12878 cells, in comparison with control cells (FIG. 16d-e). We further investigated the differentially expressed genes after knocking out this lncRNA in K562 versus in GM12878 (FIG. 15e FIG. 4e). These down-regulated genes in K562 were enriched in processes such as p53 signaling pathway and PI3K-Akt signaling pathway, which might affect cell growth and proliferation (FIG. 15f FIG. 4f, top). There were also up-regulated genes (FIG. 15f FIG. 4f, bottom), and these differentially expressed genes all contributed to the phenotypic difference of BMS1P20 knockouts in affecting cell growth between these two cell lines.

In sum, genetic perturbation of both protein-coding gene and lncRNA could be substantially enhanced by targeting splice sites. Splicing-targeting provides extra opportunity for gene disruption besides generating reading frame-shift mutations in protein-coding genes. This feature becomes irreplaceable for knocking out reading-frame-insensitive noncoding RNAs via sgRNA approach. In addition, this strategy aiming at disrupting the splice sites could be particularly useful when it is difficult to design appropriate sgRNAs targeting genes with conserved coding sequences.

CRISPR-Cas9 system has been applied to identify functional lncRNAs in large-scale through two strategies, paired-gRNA (pgRNA) deletion9 and CRISPRi¹². Although it is technically easier to scale up using CRISPRi strategy than pgRNA-mediated genomic deletion, CRISPRi as well as CRISPRa method generally act within a 1-kb window around the targeted transcriptional start site (TSS)^{12,26}, by which one would risk affecting expression of neighboring genes inadvertently for nearly 60% of lncRNA loci²⁷. Splicing-targeting strategy could effectively avoid cutting most overlapping regions using a single guide RNA, and has much better chance to avoid affecting the neighboring genes, consequently decreasing the false positive rate. After all, CRISPRi, which only decreases gene expression level instead of completely knocking out the target locus, leaves room for false-negative results.

Based on the experimental data, it is demonstrated that the new method elaborated in this invention has significant advantages in negative CRISPR screening of coding genes complementary to conventional exon-targeting method, and enables large-scale loss-of-function screen of noncoding genes using single guide RNA-CRISPR library. In addition, exon skipping or intron retention generated by splice-site disruption offers a convenient approach for functional validation of individual non-coding RNA.

### REFERENCES

1. Shalem, O. et al. Genome-scale CRISPR-Cas9 knockout screening in human cells. Science 343, 84-87 (2014).
2. Wang, T., Wei, J. J., Sabatini, D. M. & Lander, E. S. Genetic screens in human cells using the CRISPR-Cas9 system. Science 343, 80-84 (2014).
3. Koike-Yusa, H., Li, Y., Tan, E. P., Velasco-Herrera Mdel, C. & Yusa, K. Genome-wide recessive genetic screening in mammalian cells with a lentiviral CRISPR-guide RNA library. Nat Biotechnol 32, 267-273 (2014).
4. Thou, Y. et al. High-throughput screening of a CRISPR/Cas9 library for functional genomics in human cells. Nature 509, 487-491 (2014).
5. Ezkurdia, I. et al. Multiple evidence strands suggest that there may be as few as 19,000 human protein-coding genes. Hum Mol Genet 23, 5866-5878 (2014).
6. Rinn, J. L. & Chang, H. Y. Genome regulation by long noncoding RNAs. Annu Rev Biochem 81, 145-166 (2012).
7. Quinn, J. J. & Chang, H. Y. Unique features of long non-coding RNA biogenesis and function. Nat Rev Genet 17, 47-62 (2016).
8. Kretz, M. et al. Control of somatic tissue differentiation by the long non-coding RNA TINCR. Nature 493, 231-235 (2013).
9. Zhu, S. et al. Genome-scale deletion screening of human long non-coding RNAs using a paired-guide RNA CRISPR-Cas9 library. Nat Biotechnol 34, 1279-1286 (2016).
10. Guttman, M. et al. lincRNAs act in the circuitry controlling pluripotency and differentiation. Nature 477, 295-300 (2011).
11. Lin, N. et al. An evolutionarily conserved long noncoding RNA TUNA controls pluripotency and neural lineage commitment. Mol Cell 53, 1005-1019 (2014).
12. Liu, S. J. et al. CRISPRi-based genome-scale identification of functional long noncoding RNA loci in human cells. Science 355 (2017).
13. Adamson, B., Smogorzewska, A., Sigoillot, F. D., King, R. W. & Elledge, S. J. A genome-wide homologous recombination screen identifies the RNA-binding protein RBMX as a component of the DNA-damage response. Nat Cell Biol 14, 318-328 (2012).
14. Sambrook, Fritsch and Maniatis, MOLECULAR CLONING: A LABORATORY MANUAL, 2nd edition (1989).
15. F. M. Ausubel, et al. eds., CURRENT PROTOCOLS IN MOLECULAR BIOLOGY (1987).
16. M. J. MacPherson, B. D. Hames and G. R. Taylor eds., METHODS IN ENZYMOLOGY (Academic Press, Inc.): PGR 2: A PRACTICAL APPROACH (1995).
17. Harlow and Lane, eds. ANTIBODIES, A LABORATORY MANUAL, (1988).
18. R. L Freshney, ed., ANIMAL CELL CULTURE (1987).
19. Goeddel, GENE EXPRESSION TECHNOLOGY: METHODS IN ENZYMOLOGY 185, Academic Press, San Diego, Calif. (1990).
20. Seed, 1987. Nature 329: 840 (Seed, B. An LFA-3 cDNA encodes a phospholipid-linked membrane protein homologous to its receptor CD2. Nature (1987) 329: 840-842.)
21. Kaufman, et al., 1987. EMBO J. 6: 187-195 (Randal J, Kaufman, et al. Translational efficiency of polycistronic mRNAs and their utilization to express heterologous genes in mammalian cells. The EMBO Journal (1987) 6: 187-195)
22. Clancy, Suzanne. RNA Splicing: Introns, Exons and Spliceosome. Nature Education. 1, 31 (2008).
23. Black, Douglas L. Mechanisms of Alternative Pre-Messenger RNA Splicing. Annual Review of Biochemistry. 72: 291-336 (2003).
24. Ng, Bernard; Yang, Fan; et al. Increased noncanonical splicing of autoantigen transcripts provides the structural basis for expression of untolerized epitopes. Journal of Allergy and Clinical Immunology. 114: 1463-70(2004).
25. Lim, K H; Ferraris, L; et al. Using positional distribution to identify splicing elements and predict pre-mRNA processing defects in human genes. Proc. Natl. Acad. Sci. USA. 108: 11093-11098 (2011).
26. Warf, M B; Berglund, J A. Role of RNA structure in regulating pre-mRNA splicing. Trends Biochem. Sci. 35: 169-178 (2010).
27. Warf, M B; Berglund, J A. Role of RNA structure in regulating pre-mRNA splicing. Trends Biochem. Sci. 35 (3): 169-178 (2010).
28. Ren, Q. et al. A Dual-Reporter System for Real-Time Monitoring and High-throughput CRISPR/Cas9 Library Screening of the Hepatitis C Virus. Scientific reports 5, 8865 (2015).
29. Wang, T. et al. Identification and characterization of essential genes in the human genome. Science 350, 1096-1101 (2015).
30. Li, B. & Dewey, C. N. RSEM: accurate transcript quantification from RNA-Seq data with or without a reference genome. BMC bioinformatics 12, 323 (2011).
31. Leng, N. et al. EBSeq: an empirical Bayes hierarchical model for inference in RNA-seq experiments. Bioinformatics 29, 1035-1043 (2013).
32. Jiao, X. et al. DAVID-WS: a stateful web service to facilitate gene/protein list analysis. Bioinformatics 28, 1805-1806 (2012).
33. Crooks, G. E., Hon, G., Chandonia, J. M. & Brenner, S. E. WebLogo: a sequence logo generator. Genome Res 14, 1188-1190 (2004).
34. Matlin, A. J., Clark, F. & Smith, C. W. Understanding alternative splicing: towards a cellular code. Nat Rev Mol Cell Biol 6, 386-398 (2005).
35. Taggart, A. J., DeSimone, A. M., Shih, J. S., Filloux, M. E. & Fairbrother, W. G. Large-scale mapping of branchpoints in human pre-mRNA transcripts in vivo. Nat Struct Mol Biol 19, 719-721 (2012).
36. Hsu, P. D. et al. DNA targeting specificity of RNA-guided Cas9 nucleases. Nat Biotechnol 31, 827-832 (2013).
37. Xu, H. et al. Sequence determinants of improved CRISPR sgRNA design. Genome Res 25, 1147-1157 (2015).
38. Heidari, N. et al. Genome-wide map of regulatory interactions in the human genome. Genome Res 24, 1905-1917 (2014).
39. Muller, R. Y., Hammond, M. C., Rio, D. C. & Lee, Y. J. An Efficient Method for Electroporation of Small Interfering RNAs into ENCODE Project Tier 1 GM12878 and K562 Cell Lines. J Biomol Tech 26, 142-149 (2015).
40. Joung, J. et al. Genome-scale activation screen identifies a lncRNA locus regulating a gene neighbourhood. Nature (2017).
41. Goyal, A. et al. Challenges of CRISPR/Cas9 applications for long non-coding RNA genes. Nucleic Acids Res 45, e12 (2017).

1. A CRISPR/Cas guide RNA construct for disrupting a long non-coding RNA in a eukaryotic genome comprising a guide sequence targeting a genomic sequence around a splice site of a long non-coding RNA and a guide hairpin sequence, operably linked to a promoter.

2-4. (canceled)

5. The CRISPR/Cas guide RNA construct of claim 1, wherein the guide sequence targets a genomic sequence within the region spanning −50-bp to +75-bp surrounding a SD site or SA site of a long non-coding RNA.

6. The CRISPR/Cas guide RNA construct of claim 5, wherein the guide sequence targets a genomic sequence within the region spanning −30-bp to +30-bp surrounding a SD site or SA site of a long non-coding RNA.

7. The CRISPR/Cas guide RNA construct of claim 6, wherein the guide sequence targets a genomic sequence within the region spanning −10-bp to +10-bp surrounding a SD site or SA site of a long non-coding RNA.

8. The CRISPR/Cas guide RNA construct of claim 1, wherein the guide RNA construct is a viral vector or a plasmid.

9. A library composed of a plurality of the CRISPR/Cas guide RNA construct of claim 1.

10-15. (canceled)

16. A method for determining the functional profile of a long non-coding RNA comprising
introducing, into a host cell a CRISPR/Cas guide RNA construct comprising a guide sequence targeting a genomic sequence around a splice site of a long non-coding RNA and a guide hairpin sequence, operably linked to a promoter,
expressing the guide RNA that targets the genomic sequence in the host cell, and in the presence of a CRISPR/Cas nuclease, introducing exon skipping and/or intron retention in the long non-coding RNA, and thereby determining the functional profile of the long non-coding RNA.

17. The method of claim 16, wherein the guide sequence targets a genomic sequence within the region spanning −50-bp to +75-bp surrounding a SD site or SA site of a long non-coding RNA.

18. The method of claim 17, wherein the guide sequence targets a genomic sequence within the region spanning −30-bp to +30-bp surrounding a SD site or SA site of a long non-coding RNA.

19. The method of claim 18, wherein the guide sequence targets a genomic sequence within the region spanning −10-bp to +10-bp surrounding a SD site or SA site of a long non-coding RNA.

20. The method of claim 16, wherein the functional profile comprises a cellular phenotype change and/or an increase or a decrease of expression of a coding gene or non-coding gene.

21. The method of claim 20, wherein the coding gene is an exogenous reporter gene or a native coding gene in the genome.

22. The method of claim 16, wherein the host cell is in a host cell population and each host cell independently comprises a unique guide RNA construct.

23. The method of claim 22, wherein the method is a high throughput method for screening or identifying long non-coding RNAs in a eukaryotic genome.

24. (canceled)

25. A method for perturbating or eliminating the function of a long non-coding RNA in a eukaryotic cell comprising introducing into the eukaryotic cell one or more CRISPR/Cas guide RNAs that target one or more polynucleotide sequences around one or more splice sites of the long non-coding RNA, whereby the one or more guide RNAs target the one or more polynucleotide sequences around the one or more splice sites of the long non-coding RNA and in the presence of Cas protein, the one or more polynucleotide sequences are cleaved, resulting in intron retention and/or exon skipping of the long non-coding RNA and thus perturbating or eliminating the function of the long non-coding RNA.

26. The method of claim 25, the guide RNA targets a polynucleotide sequence within the region spanning −50-bp to +75-bp surrounding a SD site or SA site of a long non-coding RNA.

27. The method of claim 26, the guide RNA targets a polynucleotide sequence within the region spanning −30-bp to +30-bp surrounding a SD site or SA site of a long non-coding RNA.

28. The method of claim 27, the guide RNA targets a polynucleotide sequence within the region spanning −10-bp to +10-bp surrounding a SD site or SA site of a long non-coding RNA.

29-38. (canceled)

39. The method of claim 25, further comprising identifying the function of the lncRNA as being necessary for the growth and proliferation of tumor cells, wherein perturbating the function of the lncRNA thereby inhibits the growth and proliferation of the tumor cells.

40. The method of claim 39, wherein the lncRNAs necessary for growth and proliferation of tumor cells are selected from the group consisting of XXbac-B135H6.15, RP11-848P1.5, AC005330.2, AP001062.9, AP005135.2, RP11-867G23.4, LINC01049, DGCR5, RP11-509A17.3, CTB-25J19.1, CTD-2517M22.17, CROCCP2, AC016629.8, CTC-490G23.4, RP11-117D22.1, AC067969.2, RP11-251M1.1, AC004471.9, AC004471.10, AC002472.11, RP11-429J17.7, RP11-56N19.5, TMEM191A, LL22NC03-102D1.18, LINC00410, LL22NC03-23C6.13, RP11-83J21.3, RP11-544A12.4, ANKRD62P1-PARP4P3, CTD-2031P19.5, XXbac-B444P24.8, RP11-464F9.21, TPTEP1, MIR17HG and BMS1P20.
